(19) United States
(12) Patent Application Publication
(10) Pub. No.: US 2013/0105766 A1
(43) Pub. Date: May 2, 2013

(54) Method For Removing Halogens From An Aromatic Compound

(71) Applicants: Gregg Kottas, Ewing, NJ (US); Walter Yeager, Yardley, PA (US); Chuanjun Xia, Lawrenceville, NJ (US)

(72) Inventors: Gregg Kottas, Ewing, NJ (US); Walter Yeager, Yardley, PA (US); Chuanjun Xia, Lawrenceville, NJ (US)

(73) Assignee: Universal Display Corporation, Ewing, NJ (US)

(21) Appl. No.: 13/287,581

(22) Filed: Nov. 2, 2011

(51) Int. Cl. H01L 51/52; C07F 7/08; H01L 51/56; C07D 233/58

(52) U.S. Cl. 257/40; 548/343.5; 556/489; 438/46; 257/E51.018; 257/E51.026

(57) ABSTRACT

A method of removing halogens from an aromatic compound is disclosed. The method is compatible with a wide variety of functional groups, and avoids the use of highly reactive reagents such as organolithium compounds. Additionally, the dehalogenated compounds are separable from reaction by-products on silica gel. Compounds dehalogenated by the method described herein are suitable for incorporation into OLEDs and other electronic devices.

The claimed invention was made by, on behalf of, and/or in connection with one or more of the following parties to a joint university corporation research agreement: Regents of the University of Michigan, Princeton University, The University of Southern California, and the Universal Display Corporation. The agreement was in effect on and before the date the claimed invention was made, and the claimed invention was made as a result of activities undertaken within the scope of the agreement.

### FIELD OF THE INVENTION

The present invention relates to methods of removing halogens from a chemical substances, and in particular to reducing the halogen content of aromatic compounds.

### BACKGROUND

Opto-electronic devices that make use of organic materials are becoming increasingly desirable for a number of reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting devices (OLEDs), organic phototransistors, organic photovoltaic cells, and organic photodetectors. For OLEDs, the organic materials may have performance advantages over conventional materials. For example, the wavelength at which an organic emissive layer emits light may generally be readily tuned with appropriate dopants.

OLEDs make use of thin organic films that emit light when voltage is applied across the device. OLEDs are becoming an increasingly interesting technology for use in applications such as flat panel displays, illumination, and backlighting. Several OLED materials and configurations are described in U.S. Pat. Nos. 5,844,363, 6,303,238, and 5,707,745, which are incorporated herein by reference in their entirety.

One application for phosphorescent emissive molecules is a full color display. Industry standards for such a display call for pixels adapted to emit particular colors, referred to as “saturated” colors. In particular, these standards call for saturated red, green, and blue pixels. Color may be measured using CIE coordinates, which are well known to the art.

One example of a green emissive molecule is tris(2-phenylpyridine)iridium, denoted Ir(ppy)₃, which has the following structure:

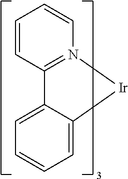

In this, and later figures herein, we depict the dative bond from nitrogen to metal (here, Ir) as a straight line.

As used herein, the term “organic” includes polymeric materials as well as small molecule organic materials that may be used to fabricate organic opto-electronic devices. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety. The core moiety of a dendrimer may be a fluorescent or phosphorescent small molecule emitter. A dendrimer may be a “small molecule,” and it is believed that all dendrimers currently used in the field of OLEDs are small molecules.

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

More details on OLEDs, and the definitions described above, can be found in U.S. Pat. No. 7,279,704, which is incorporated herein by reference in its entirety.

### SUMMARY OF THE INVENTION

In one aspect a method of reducing halogen content is provided. The method comprises a) reacting at least one aromatic halide with a transition metal complex by 1) oxidatively inserting the transition metal complex into a carbon-halogen bond in said aromatic halide to form an metal-carbon bonded species; and 2) converting the metal-carbon bonded species into a reaction product, wherein the reaction product has the halogen in the aromatic halide replaced by hydrogen. The oxidative insertion takes place in the presence of a base. The method further comprises b) purifying the reaction product to give a purified product.

In one aspect, the method further comprises using the purified product in an organic electronic device further comprising depositing a first electrode, depositing an organic layer comprising the purified product on the first electrode, and depositing a second electrode on the organic layer.

In one aspect, the purified product contains less than 300 ppm of halogen. In one aspect, the purified product contains less than 50 ppm of halogen. In another aspect, the purified product contains less than 10 ppm of halogen.

In one aspect, the transition metal complex comprises a metal complex containing palladium, platinum, copper, or nickel.

In one aspect the aromatic halide comprises an imidazole, a pyridine, a quinoline, an isoquinoline, an aldehyde, a ketone, an alcohol, or combinations thereof. In another aspect, the aromatic halide comprises a triphenylene, an aza-triphenylene, a carbazole, an aza-carbazole, a dibenzothiphene, a dibenzofuran, a dibenzoselenophene, an aza-dibenzothiophene, an aza-dibenzofuran, an aza-dibenzoselenophene, or combinations thereof.

In one aspect, less than 10 ppm of the reaction product contains a new carbon-carbon bond. In another aspect, less than 90% by weight of the reaction product contains a new carbon-carbon bond. In one aspect, the aromatic halide is used in an electroluminescent device.

In one aspect, the transition metal complex is a palladium complex. In another aspect, the palladium complex is a palladium(II) complex. In one aspect, the palladium(II) complex is selected from the group consisting of PdCl₂, Pd(PPh₃)₂Cl₂, Pd(OAc)₂, [Pd(allyl)Cl]₂, Pd(benzonitrile)₂O₂, Pd(dppf)Cl₂, and combinations thereof.

In one aspect, the palladium complex includes one or more ligands selected from the group consisting of dibenzylideneacetone,

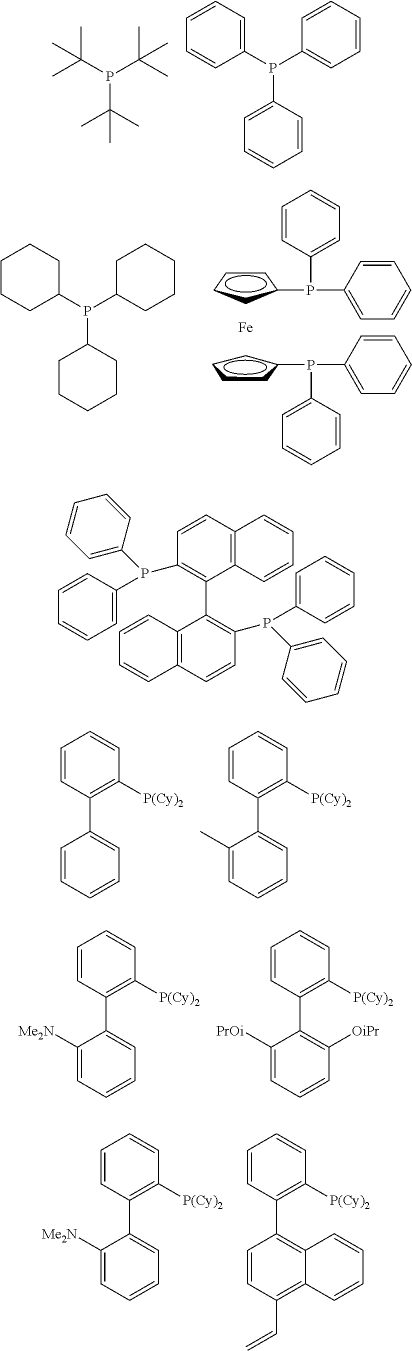

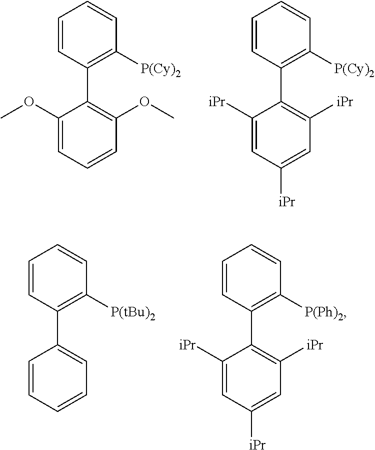

and combinations thereof.

In one aspect, the base is an oxygen-containing base. In one aspect, the base is an alkali metal salt of a compound selected from the group consisting of an alkoxide, a phosphate, and a carbonate. In on aspect, the alkoxide is NaO^{t}Bu or KO^{t}Bu. In another aspect, the phosphate is K₃PO₄.

In one aspect, a first device is provided. In one aspect, the first device comprises a first organic light emitting device, and further comprises an anode, a cathode, and an organic layer, disposed between the anode and the cathode, wherein the organic layer is deposited from a source comprising a purified product formed by reacting at least one aromatic halide with a transition metal complex by 1) oxidatively inserting the transition metal complex into a carbon-halogen bond in said aromatic halide to form an metal-carbon bonded species, and 2) converting the metal-carbon bonded species into a reaction product, wherein the reaction product is where the halogen in the aromatic halide is replaced by hydrogen. The oxidative insertion takes place in the presence of a base. Purifying the reaction product results in the formation of the purified product. In one aspect, the organic layer has a halogen content of less than 300 ppm.

### BRIEF DESCRIPTION OF THE DRAWINGS

FIG. 1 shows an organic light emitting device.

FIG. 2 shows an inverted organic light emitting device that does not have a separate electron transport layer.

FIG. 3 shows an exemplary dehalogenation scheme according to the method described herein.

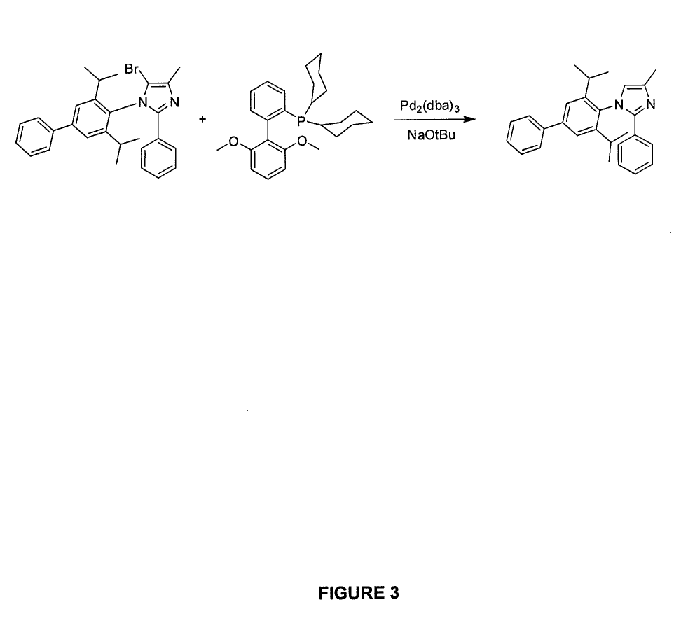

### DETAILED DESCRIPTION

Generally, an OLED comprises at least one organic layer disposed between and electrically connected to an anode and a cathode. When a current is applied, the anode injects holes and the cathode injects electrons into the organic layer(s). The injected holes and electrons each migrate toward the oppositely charged electrode. When an electron and hole localize on the same molecule, an “exciton,” which is a localized electron-hole pair having an excited energy state, is formed. Light is emitted when the exciton relaxes via a photoemissive mechanism. In some cases, the exciton may be localized on an excimer or an exciplex. Non-radiative mechanisms, such as thermal relaxation, may also occur, but are generally considered undesirable.

The initial OLEDs used emissive molecules that emitted light from their singlet states (“fluorescence”) as disclosed, for example, in U.S. Pat. No. 4,769,292, which is incorporated by reference in its entirety. Fluorescent emission generally occurs in a time frame of less than 10 nanoseconds.

More recently, OLEDs having emissive materials that emit light from triplet states (“phosphorescence”) have been demonstrated. Baldo et al., “Highly Efficient Phosphorescent Emission from Organic Electroluminescent Devices,” Nature, vol. 395, 151-154, 1998; (“Baldo-I”) and Baldo et al., “Very high-efficiency green organic light-emitting devices based on electrophosphorescence,” Appl. Phys. Lett., vol. 75, No. 3, 4-6 (1999) (“Baldo-II”), which are incorporated by reference in their entireties. Phosphorescence is described in more detail in U.S. Pat. No. 7,279,704 at cols. 5-6, which are incorporated by reference.

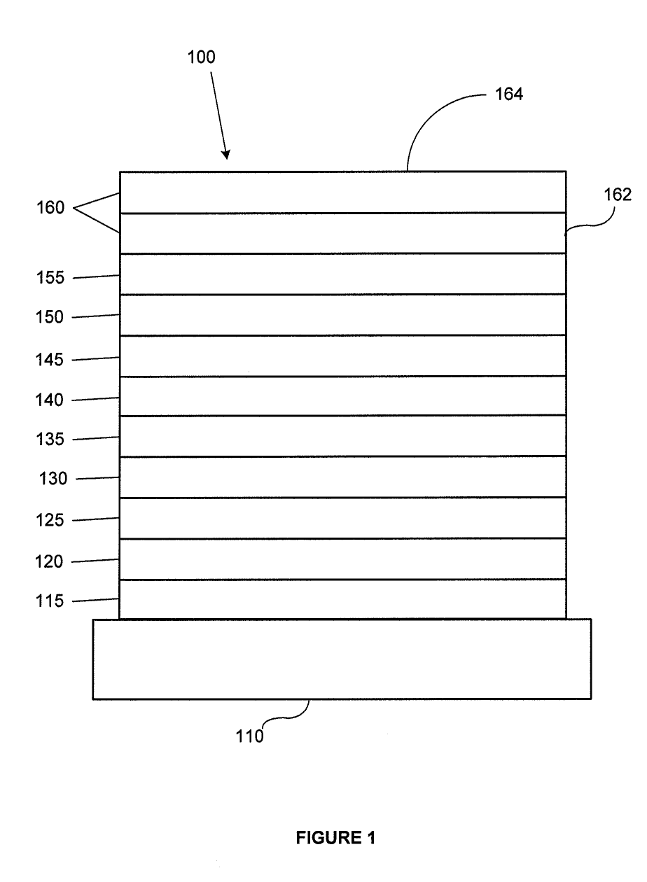

FIG. 1 shows an organic light emitting device 100. The figures are not necessarily drawn to scale. Device 100 may include a substrate 110, an anode 115, a hole injection layer 120, a hole transport layer 125, an electron blocking layer 130, an emissive layer 135, a hole blocking layer 140, an electron transport layer 145, an electron injection layer 150, a protective layer 155, and a cathode 160. Cathode 160 is a compound cathode having a first conductive layer 162 and a second conductive layer 164. Device 100 may be fabricated by depositing the layers described, in order. The properties and functions of these various layers, as well as example materials, are described in more detail in U.S. Pat. No. 7,279,704 at cols. 6-10, which are incorporated by reference.

More examples for each of these layers are available. For example, a flexible and transparent substrate-anode combination is disclosed in U.S. Pat. No. 5,844,363, which is incorporated by reference in its entirety. An example of a p-doped hole transport layer is m-MTDATA doped with F.sub.4-TCNQ at a molar ratio of 50:1, as disclosed in U.S. Patent Application Publication No. 2003/0230980, which is incorporated by reference in its entirety. Examples of emissive and host materials are disclosed in U.S. Pat. No. 6,303,238 to Thompson et al., which is incorporated by reference in its entirety. An example of an n-doped electron transport layer is BPhen doped with Li at a molar ratio of 1:1, as disclosed in U.S. Patent Application Publication No. 2003/0230980, which is incorporated by reference in its entirety. U.S. Pat. Nos. 5,703,436 and 5,707,745, which are incorporated by reference in their entireties, disclose examples of cathodes including compound cathodes having a thin layer of metal such as Mg:Ag with an overlying transparent, electrically-conductive, sputter-deposited ITO layer. The theory and use of blocking layers is described in more detail in U.S. Pat. No. 6,097,147 and U.S. Patent Application Publication No. 2003/0230980, which are incorporated by reference in their entireties. Examples of injection layers are provided in U.S. Patent Application Publication No. 2004/0174116, which is incorporated by reference in its entirety. A description of protective layers may be found in U.S. Patent Application Publication No. 2004/0174116, which is incorporated by reference in its entirety.

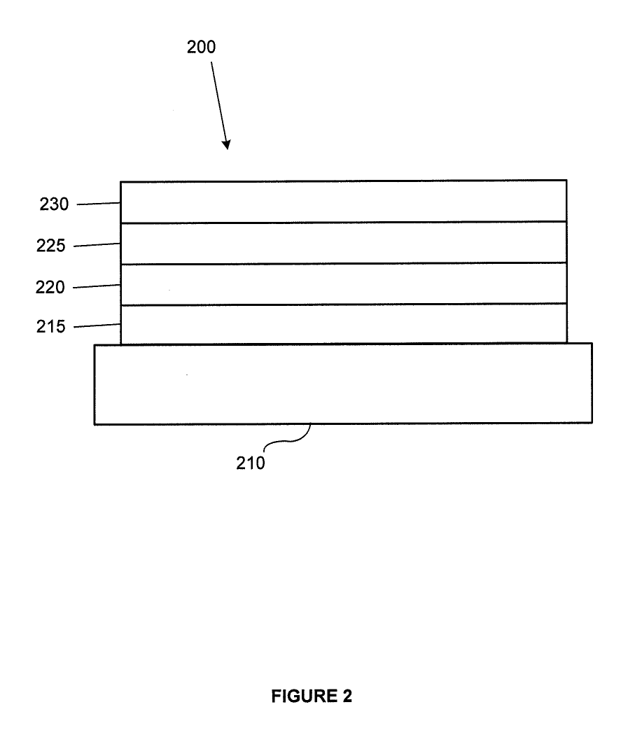

FIG. 2 shows an inverted OLED 200. The device includes a substrate 210, a cathode 215, an emissive layer 220, a hole transport layer 225, and an anode 230. Device 200 may be fabricated by depositing the layers described, in order. Because the most common OLED configuration has a cathode disposed over the anode, and device 200 has cathode 215 disposed under anode 230, device 200 may be referred to as an “inverted” OLED. Materials similar to those described with respect to device 100 may be used in the corresponding layers of device 200. FIG. 2 provides one example of how some layers may be omitted from the structure of device 100.

The simple layered structure illustrated in FIGS. 1 and 2 is provided by way of non-limiting example, and it is understood that embodiments of the invention may be used in connection with a wide variety of other structures. The specific materials and structures described are exemplary in nature, and other materials and structures may be used. Functional OLEDs may be achieved by combining the various layers described in different ways, or layers may be omitted entirely, based on design, performance, and cost factors. Other layers not specifically described may also be included. Materials other than those specifically described may be used. Although many of the examples provided herein describe various layers as comprising a single material, it is understood that combinations of materials, such as a mixture of host and dopant, or more generally a mixture, may be used. Also, the layers may have various sublayers. The names given to the various layers herein are not intended to be strictly limiting. For example, in device 200, hole transport layer 225 transports holes and injects holes into emissive layer 220, and may be described as a hole transport layer or a hole injection layer. In one embodiment, an OLED may be described as having an “organic layer” disposed between a cathode and an anode. This organic layer may comprise a single layer, or may further comprise multiple layers of different organic materials as described, for example, with respect to FIGS. 1 and 2.

Structures and materials not specifically described may also be used, such as OLEDs comprised of polymeric materials (PLEDs) such as disclosed in U.S. Pat. No. 5,247,190 to Friend et al., which is incorporated by reference in its entirety. By way of further example, OLEDs having a single organic layer may be used. OLEDs may be stacked, for example as described in U.S. Pat. No. 5,707,745 to Forrest et al, which is incorporated by reference in its entirety. The OLED structure may deviate from the simple layered structure illustrated in FIGS. 1 and 2. For example, the substrate may include an angled reflective surface to improve out-coupling, such as a mesa structure as described in U.S. Pat. No. 6,091,195 to Forrest et al., and/or a pit structure as described in U.S. Pat. No. 5,834,893 to Bulovic et al., which are incorporated by reference in their entireties.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. patent application Ser. No. 10/233,470, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink jet and OVJD. Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution proccessability than those having symmetric structures, because asymmetric materials may have a lower tendency to recrystallize. Dendrimer substituents may be used to enhance the ability of small molecules to undergo solution processing.

Devices fabricated in accordance with embodiments of the invention may be incorporated into a wide variety of consumer products, including flat panel displays, computer monitors, televisions, billboards, lights for interior or exterior illumination and/or signaling, heads up displays, fully transparent displays, flexible displays, laser printers, telephones, cell phones, personal digital assistants (PDAs), laptop computers, digital cameras, camcorders, viewfinders, micro-displays, vehicles, a large area wall, theater or stadium screen, or a sign. Various control mechanisms may be used to control devices fabricated in accordance with the present invention, including passive matrix and active matrix. Many of the devices are intended for use in a temperature range comfortable to humans, such as 18 degrees C. to 30 degrees C., and more preferably at room temperature (20-25 degrees C.).

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

The terms halo, halogen, alkyl, cycloalkyl, alkenyl, alkynyl, arylkyl, heterocyclic group, aryl, aromatic group, and heteroaryl are known to the art, and are defined in U.S. Pat. No. 7,279,704 at cols. 31-32, which are incorporated herein by reference.

In one embodiment a method of reducing halogen content is provided. The method comprises a) reacting at least one aromatic halide with a transition metal complex by 1) oxidatively inserting the transition metal complex into a carbon-halogen bond in said aromatic halide to form an metal-carbon bonded species; and 2) converting the metal-carbon bonded species into a reaction product, wherein the reaction product has the halogen in the aromatic halide replaced by hydrogen. The oxidative insertion takes place in the presence of a base. The method further comprises b) purifying the reaction product to give a purified product. By “oxidatively inserting,” it is meant that a suitable transition metal complex reacts in such a way as to be placed in between a carbon-halogen bond, where the transition metal becomes bonded to the carbon atom formerly bearing the halogen, and the halogen becomes attached to the transition metal. The mechanism for oxidative addition or insertion is well known to those of skill in the art. The “metal-carbon bonded species” is the complex that results from the oxidative insertion, and as described above, contains a bond between the carbon atom formerly bearing a halogen and a transition metal.

The deleterious role of halogens in OLED devices has been known for some time, and have been reported in, for example, Fleissner, A.; et al. Chem. Mater. 2009, 21, 4288 and Andersson, G.; et al. Synth. Met. 2000, 113, 245, yet halogenated intermediates are common in the synthesis of OLED materials. Accordingly, there exists a need for an effective and economic method of removing halogens that preferably does not use highly reactive reagents that require special handling, for example.

A variety of methods exist for dehalogenating aryl halides, including the use of Friedel-Crafts catalysts (e.g., AlCl₃), reducing agents (e.g., tributyltin hydride, iron pentacarbonyl, sodium-mercury amalgam, lithium aluminum hydride, sodium borohydride, sodium hydride), as well as photochemical and electrochemical reduction. Other common methods for the reduction of aryl halides are through the formation of organometallic complexes such as aryllithium (e.g. ArLi) species and arylmagnesium (“Grignard”) species (e.g. ArMgX, where X is a halogen, typically bromine) followed by protonation with a proton source:

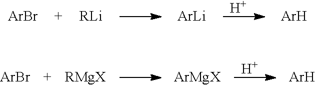

Dehalogenation using organolithium or Grignard reagents has a number of disadvantages, including typically requiring very low reaction temperatures (e.g. −78° C.), and having limited functional group compatibility due to their high reactivity with many important and useful organic functional groups such as ketones, aldehydes, etc. Additionally, because ogranolithium and Grignard reagents are highly reactive, they also require special handling procedures to prevent them from coming into contact with water, for example.

As discussed above, halogenated impurities in OLED devices are undesirable. The method of dehalogenating aromatic compounds disclosed herein is suitable for preparing compounds that can be used in a variety of OLED devices disclosed herein and otherwise known to one or ordinary skill in the art. In one embodiment, the method further comprises using the purified product in an organic electronic device further comprising depositing a first electrode, depositing an organic layer comprising the purified product on the first electrode, and depositing a second electrode on the organic layer.

One of ordinary skill in the art will appreciate that compound structure can often dictate reactivity and reaction time. For example, hindered compounds tend to require longer reaction times. Advantageously, the method disclosed herein is suitable for dehalogenating even highly hindered compounds, i.e. those with significant steric crowding near the carbon or carbons to which the halogen is attached to, which may be caused by, for example, bulky groups such as tert-butyl, iso-propyl, phenyl, etc. By use of the method disclosed herein, the amount of halogen impurity decreases over time. Table 1 lists the percentage of halogenated impurity as a function of time for dehalogenation of diphenyldi(triphenylen-2-yl)silane containing 1.1% brominated impurity, which is detailed in the Experimental section below. After 96 hours of reaction time, the halogen content was below 300 ppm as measured by HPLC. An iterative process can be performed whereby, for example, a composition having less than 300 ppm halogenated impurity, is subjected to additional rounds of purification by the method disclosed herein to achieve the desired level of purity.

In one embodiment, the purified product contains less than 300 ppm of halogen. In one embodiment, the purified product contains less than 50 ppm of halogen. In another embodiment, the purified product contains less than 10 ppm of halogen.

TABLE 1
Percentage halogenated impurity as a function of time.

| | Percent brominated |
|---|---|
| Time (hours) | impurity (%) |
| 24 | 0.073 |
| 36 | 0.056 |
| 72 | 0.048 |
| 96 | 0.029 |

In one embodiment, the transition metal complex comprises a metal complex containing palladium, platinum, copper, or nickel. Aryl palladium complexes are often used in cross coupling reactions such as the Heck Reaction, the Suzuki coupling, the Stille coupling, the Negishi coupling, the Sonogashira reaction and the Buchwald-Hartwig coupling. In these instances, the aryl-palladium species then goes onto couple with a reactive partner such as a boronic acid (Suzuki), a tin reagent (Stille), a zinc reagent (Negishi) or an acetylide (Sonogashira), typically another aryl unit, through reductive elimination to form a new species:

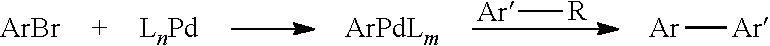

In one embodiment the aromatic halide comprises an imidazole, a pyridine, a quinoline, an isoquinoline, an aldehyde, a ketone, an alcohol, or combinations thereof. In another embodiment, the aromatic halide comprises a triphenylene, an aza-triphenylene, a carbazole, an aza-carbazole, a dibenzothiphene, a dibenzofuran, a dibenzoselenophene, an aza-dibenzothiophene, an aza-dibenzofuran, an aza-dibenzoselenophene, or combinations thereof. The term “aza” means that one or more C—H groups in the corresponding molecule or fragment can be replaced by nitrogen.

In contrast to prior art dehalogenations that utilize cross-coupling reactions to form carbon-carbon bonds to remove halogenated impurities, e.g. Suzuki coupling with aryl boronic acids, the dehalogenation method described herein advantageously does not form additional carbon-carbon bonds. Procedures using the Suzuki coupling/reaction are disclosed in, for example, US2007/060777 and EP1985643A. When a halogen is removed from a molecule by replacing it with a new carbon-carbon bond, as in the prior art, instead of hydrogen, as in the method disclosed herein, the product containing a new carbon-carbon bond can become difficult to separate from the desired material. Additionally, for host materials used in PHOLEDs, introducing an extra phenyl (or aryl) group can increase conjugation and lower the triplet energy of the compound, which is not desirable in many cases, such as for high triplet energy emitters. In one embodiment, less than 10 ppm of the reaction product contains a new carbon-carbon bond. In another embodiment, less than 90% by weight of the reaction product contains a new carbon-carbon bond. In one embodiment, the aromatic halide is used in an electroluminescent device.

In one embodiment, the transition metal complex is a palladium complex. In another embodiment, the palladium complex is a palladium(II) complex. Complexes that contain phosphine ligands have the phosphine ligand coordinated to the palladium (or other transition metal) center through the phosphorus atom. In one embodiment, the palladium(II) complex is selected from the group consisting of PdCl₂, Pd(PPh₃)₂Cl₂, Pd(OAc)₂, [Pd(allyl)Cl]₂, Pd(benzonitrile)₂Cl₂, Pd(dppf)Cl₂, and combinations thereof.

In one embodiment, the palladium complex includes one or more ligands selected from the group consisting of dibenzylideneacetone (dba),

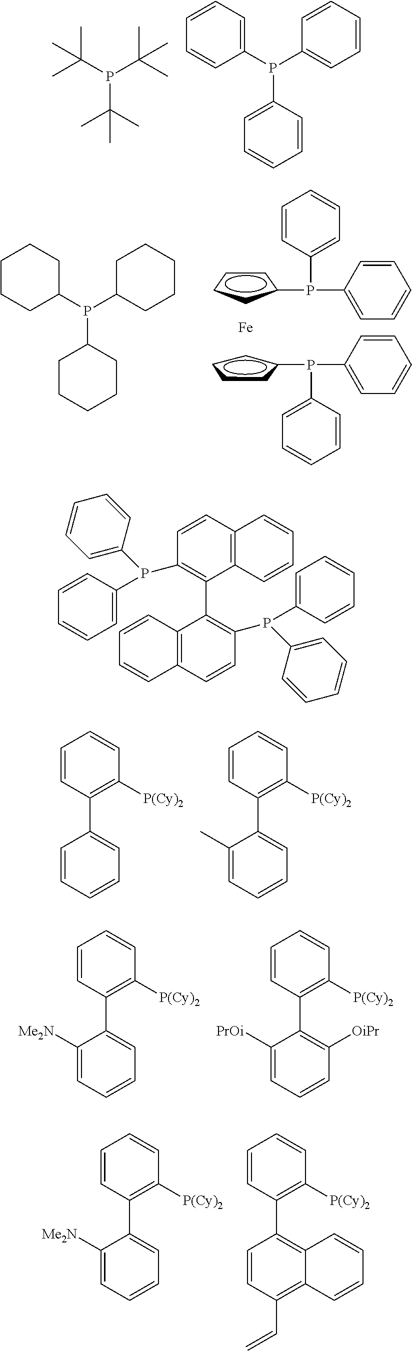

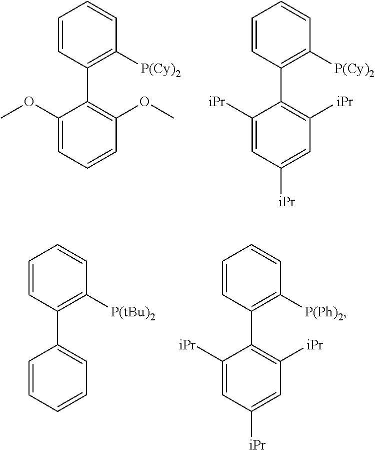

and combinations thereof. It should be understood by one of ordinary skill in the art that the palladium (or other transition metal) center may have other ligands coordinated to it in addition to the ligands disclosed herein. For example, the palladium (or other transition metal) complex or metal-carbon bonded species may also have solvent or substrate molecules coordinated to it.

In one embodiment, the base is an oxygen-containing base. In one embodiment, the base is an alkali metal salt of a compound selected from the group consisting of an alkoxide, a phosphate, and a carbonate. In one embodiment, the alkoxide is NaO^{t}Bu or KO^{t}Bu. In another embodiment, the phosphate is K₃PO₄.

In one embodiment, a first device is provided. In one embodiment, the first device comprises a first organic light emitting device, and further comprises an anode, a cathode, and an organic layer, disposed between the anode and the cathode, wherein the organic layer is deposited from a source comprising a purified product formed by a) reacting at least one aromatic halide with a transition metal complex by 1) oxidatively inserting the transition metal complex into a carbon-halogen bond in said aromatic halide to form an metal-carbon bonded species, and 2) converting the metal-carbon bonded species into a reaction product, wherein the reaction product is where the halogen in the aromatic halide is replaced by hydrogen. The oxidative insertion takes place in the presence of a base. Purifying the reaction product results in the formation of the purified product. In one embodiment, the organic layer has a halogen content of less than 300 ppm.

Combination with Other Materials

The materials described herein as useful for a particular layer in an organic light emitting device may be used in combination with a wide variety of other materials present in the device. For example, emissive dopants disclosed herein may be used in conjunction with a wide variety of hosts, transport layers, blocking layers, injection layers, electrodes and other layers that may be present. The materials described or referred to below are non-limiting examples of materials that may be useful in combination with the compounds disclosed herein, and one of skill in the art can readily consult the literature to identify other materials that may be useful in combination.

### HIL/HTL:

A hole injecting/transporting material to be used in the present invention is not particularly limited, and any compound may be used as long as the compound is typically used as a hole injecting/transporting material. Examples of the material include, but not limit to: a phthalocyanine or porphryin derivative; an aromatic amine derivative; an indolocarbazole derivative; a polymer containing fluorohydrocarbon; a polymer with conductivity dopants; a conducting polymer, such as PEDOT/PSS; a self-assembly monomer derived from compounds such as phosphonic acid and silane derivatives; a metal oxide derivative, such as MoOₓ; a p-type semiconducting organic compound, such as 1,4,5,8,9,12-Hexaazatriphenylenehexacarbonitrile; a metal complex, and a cross-linkable compounds.

Examples of aromatic amine derivatives used in HIL or HTL include, but not limit to the following general structures:

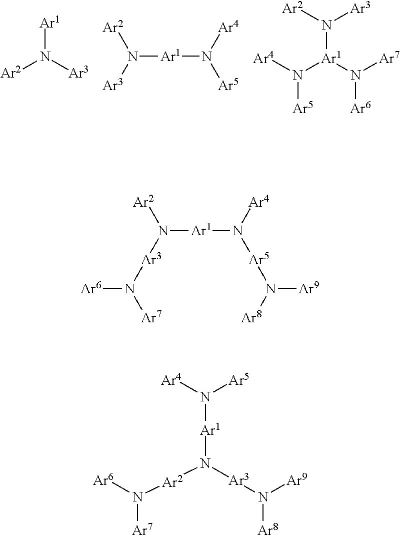

Each of Ar¹ to Ar⁹ is selected from the group consisting aromatic hydrocarbon cyclic compounds such as benzene, biphenyl, triphenyl, triphenylene, naphthalene, anthracene, phenalene, phenanthrene, fluorene, pyrene, chrysene, perylene, azulene; group consisting aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine; and group consisting 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Wherein each Ar is further substituted by a substituent selected from the group consisting of hydrogen, deuterium, halide, alkyl, cycloalkyl, heteroalkyl, arylalkyl, alkoxy, aryloxy, amino, silyl, alkenyl, cycloalkenyl, heteroalkenyl, alkynyl, aryl, heteroaryl, acyl, carbonyl, carboxylic acids, ester, nitrile, isonitrile, sulfanyl, sulfinyl, sulfonyl, phosphino, and combinations thereof.

In one aspect, Ar¹ to Ar⁹ is independently selected from the group consisting of:

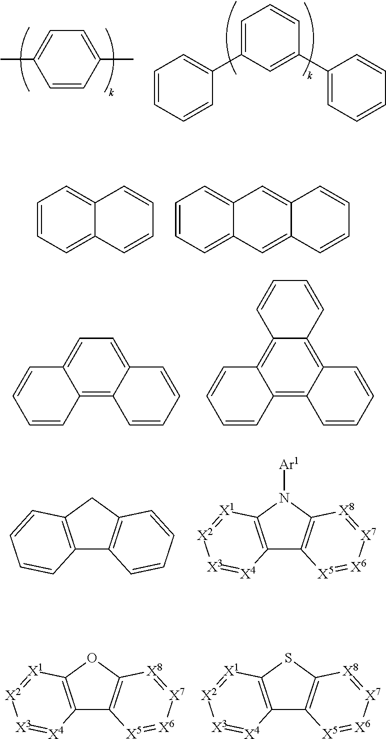

k is an integer from 1 to 20; X¹ to X⁸ is C (including CH) or N; Ar¹ has the same group defined above.

Examples of metal complexes used in HIL or HTL include, but not limit to the following general formula:

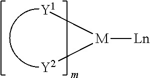

M is a metal, having an atomic weight greater than 40; (Y¹—Y²) is a bidentate ligand, Y¹ and Y² are independently selected from C, N, O, P, and S; L is an ancillary ligand; m is an integer value from 1 to the maximum number of ligands that may be attached to the metal; and m+n is the maximum number of ligands that may be attached to the metal.

In one aspect, (Y¹—Y²) is a 2-phenylpyridine derivative.

In another aspect, (Y¹—Y²) is a carbene ligand.

In another aspect, M is selected from Ir, Pt, Os, and Zn.

In a further aspect, the metal complex has a smallest oxidation potential in solution vs. Fc⁺/Fc couple less than about 0.6 V.

### Host:

The light emitting layer of the organic EL device of the present invention preferably contains at least a metal complex as light emitting material, and may contain a host material using the metal complex as a dopant material. Examples of the host material are not particularly limited, and any metal complexes or organic compounds may be used as long as the triplet energy of the host is larger than that of the dopant.

Examples of metal complexes used as host are preferred to have the following general formula:

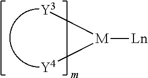

M is a metal; (Y³—Y⁴) is a bidentate ligand, Y³ and Y⁴ are independently selected from C, N, O, P, and S; L is an ancillary ligand; m is an integer value from 1 to the maximum number of ligands that may be attached to the metal; and m+n is the maximum number of ligands that may be attached to the metal.

In one aspect, the metal complexes are:

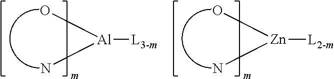

(O—N) is a bidentate ligand, having metal coordinated to atoms O and N.

In another aspect, M is selected from Ir and Pt.

In a further aspect, (Y³—Y⁴) is a carbene ligand.

Examples of organic compounds used as host are selected from the group consisting aromatic hydrocarbon cyclic compounds such as benzene, biphenyl, triphenyl, triphenylene, naphthalene, anthracene, phenalene, phenanthrene, fluorene, pyrene, chrysene, perylene, azulene; group consisting aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine; and group consisting 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Wherein each group is further substituted by a substituent selected from the group consisting of hydrogen, deuterium, halide, alkyl, cycloalkyl, heteroalkyl, arylalkyl, alkoxy, aryloxy, amino, silyl, alkenyl, cycloalkenyl, heteroalkenyl, alkynyl, aryl, heteroaryl, acyl, carbonyl, carboxylic acids, ester, nitrile, isonitrile, sulfanyl, sulfinyl, sulfonyl, phosphino, and combinations thereof.

In one aspect, host compound contains at least one of the following groups in the molecule:

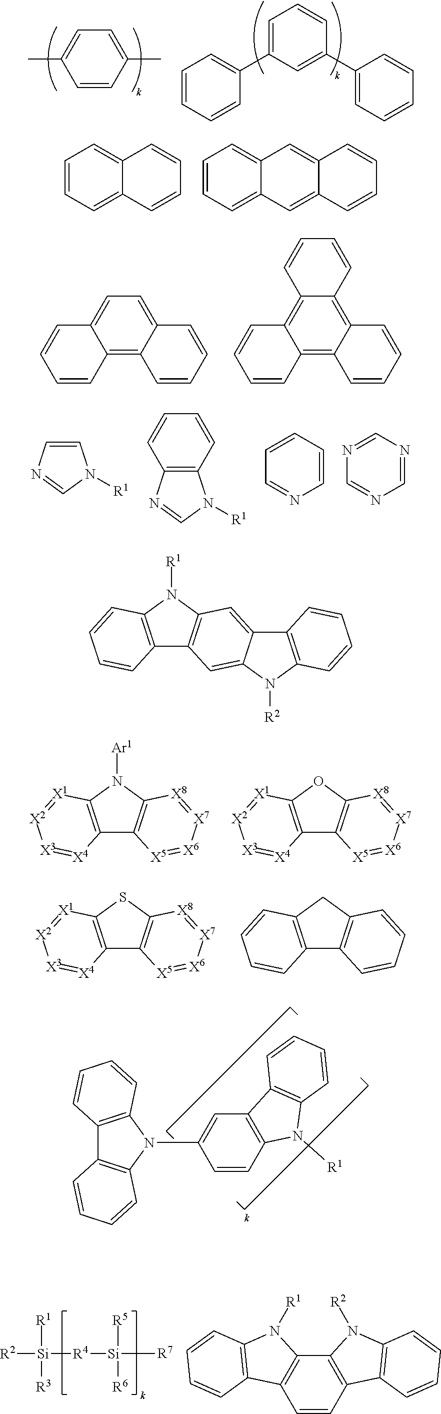

R¹ to R⁷ is independently selected from the group consisting of hydrogen, deuterium, halide, alkyl, cycloalkyl, heteroalkyl, arylalkyl, alkoxy, aryloxy, amino, silyl, alkenyl, cycloalkenyl, heteroalkenyl, alkynyl, aryl, heteroaryl, acyl, carbonyl, carboxylic acids, ester, nitrile, isonitrile, sulfanyl, sulfinyl, sulfonyl, phosphino, and combinations thereof, when it is aryl or heteroaryl, it has the similar definition as Ar's mentioned above.

k is an integer from 0 to 20.

X¹ to X⁸ is selected from C (including CH) or N.

### HBL:

A hole blocking layer (HBL) may be used to reduce the number of holes and/or excitons that leave the emissive layer. The presence of such a blocking layer in a device may result in substantially higher efficiencies as compared to a similar device lacking a blocking layer. Also, a blocking layer may be used to confine emission to a desired region of an OLED.

In one aspect, compound used in HBL contains the same molecule used as host described above.

In another aspect, compound used in HBL contains at least one of the following groups in the molecule:

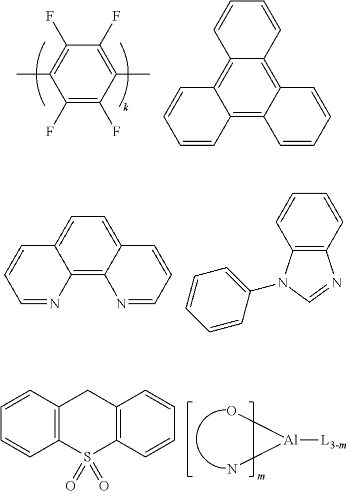

k is an integer from 0 to 20; L is an ancillary ligand, m is an integer from 1 to 3.

### ETL:

Electron transport layer (ETL) may include a material capable of transporting electrons. Electron transport layer may be intrinsic (undoped), or doped. Doping may be used to enhance conductivity. Examples of the ETL material are not particularly limited, and any metal complexes or organic compounds may be used as long as they are typically used to transport electrons.

In one aspect, compound used in ETL contains at least one of the following groups in the molecule:

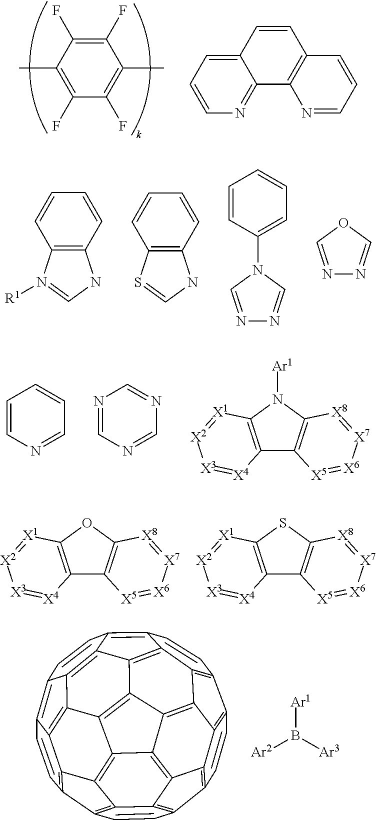

R¹ is selected from the group consisting of hydrogen, deuterium, halide, alkyl, cycloalkyl, heteroalkyl, arylalkyl, alkoxy, aryloxy, amino, silyl, alkenyl, cycloalkenyl, heteroalkenyl, alkynyl, aryl, heteroaryl, acyl, carbonyl, carboxylic acids, ester, nitrile, isonitrile, sulfanyl, sulfinyl, sulfonyl, phosphino, and combinations thereof, when it is aryl or heteroaryl, it has the similar definition as Ar's mentioned above.

Ar¹ to Ar³ has the similar definition as Ar's mentioned above.

k is an integer from 0 to 20.

X¹ to X⁸ is selected from C (including CH) or N.

In another aspect, the metal complexes used in ETL contains, but not limit to the following general formula:

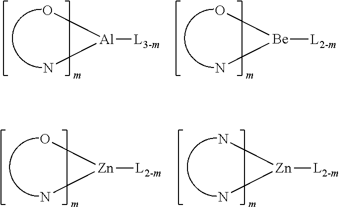

(O—N) or (N—N) is a bidentate ligand, having metal coordinated to atoms O, N or N, N; L is an ancillary ligand; m is an integer value from 1 to the maximum number of ligands that may be attached to the metal.

In any above-mentioned compounds used in each layer of the OLED device, the hydrogen atoms can be partially or fully deuterated.

In addition to and/or in combination with the materials disclosed herein, many hole injection materials, hole transporting materials, host materials, dopant materials, exciton/hole blocking layer materials, electron transporting and electron injecting materials may be used in an OLED. Non-limiting examples of the materials that may be used in an OLED in combination with materials disclosed herein are listed in Table 2 below. Table 2 lists non-limiting classes of materials, non-limiting examples of compounds for each class, and references that disclose the materials.

TABLE 2

| MATERIAL | EXAMPLES OF MATERIAL | PUBLICATIONS |
|---|---|---|
| Hole injection | | |
| materials | | |
| Phthalocyanine and porphyrin compounds | 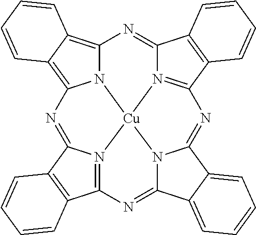 | Appl. Phys. Lett. 69, 2160 (1996) |
| Starburst triarylamines | 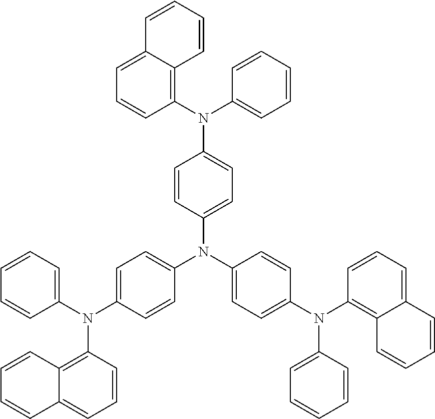 [SMILES: c1ccc(N(c2ccc(N(c3ccc(N(c4ccccc4)c4cccc5ccccc45)cc3)c3ccc(N(c4ccccc4)c4cccc5ccccc45)cc3)cc2)c2cccc3ccccc23)cc1] | J. Lumin. 72-74, 985 (1997) |
| CFₓ Fluorohydro- carbon polymer | 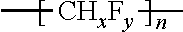 | Appl. Phys. Lett. 78, 673 (2001) |
| Conducting polymers (e.g., PEDOT: PSS, polyaniline, polypthio- phene) | 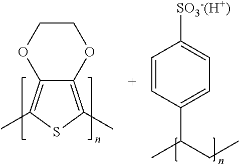 | Synth. Met. 87, 171 (1997) WO2007002683 |
| Phosphonic acid and sliane SAMs | 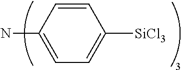 | US20030162053 |
| Triarylamine or polythiophene polymers with conductivity dopants | 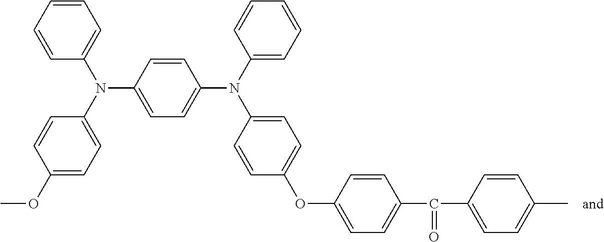 | EP1725079A1 |
| | 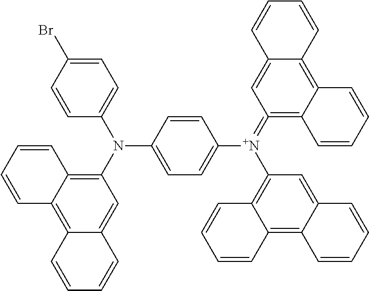 | |
| | 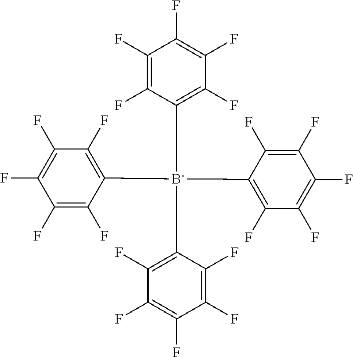 [SMILES: Fc1c(F)c(F)c([B-](c2c(F)c(F)c(F)c(F)c2F)(c2c(F)c(F)c(F)c(F)c2F)c2c(F)c(F)c(F)c(F)c2F)c(F)c1F] | |
| Arylamines complexed with metal oxides such as molybdenum and tungsten oxides | 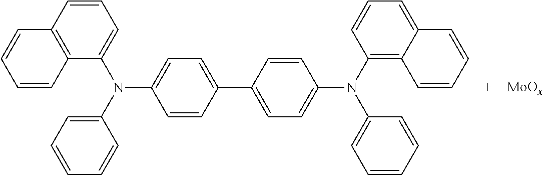 [SMILES: c1ccc(N(c2ccc(-c3ccc(N(c4ccccc4)c4cccc5ccccc45)cc3)cc2)c2cccc3ccccc23)cc1 ; "MoOx"] | SID Symposium Digest, 37, 923 (2006) WO2009018009 |
| p-type semiconducting organic complexes | 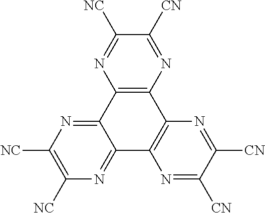 [SMILES: N#Cc1nc2c3nc(C#N)c(C#N)nc3c3nc(C#N)c(C#N)nc3c2nc1C#N] | US20020158242 |
| Metal organometallic complexes | 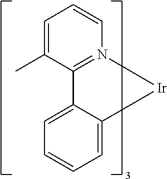 | US20060240279 |
| Cross-linkable compounds | 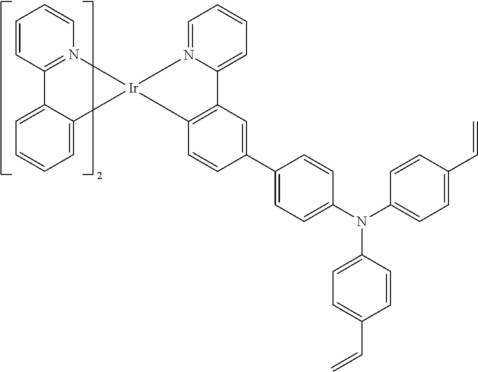 | US20080220265 |
| Hole | | |
| transporting | | |
| materials | | |
| Triarylamines (e.g., TPD, α- NPD) | 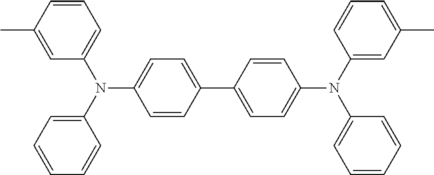 | Appl. Phys. Lett. 51, 913 (1987) |
| | 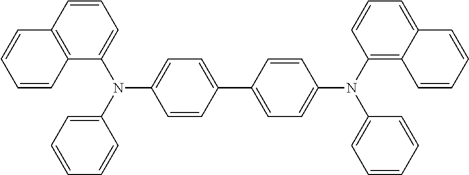 [SMILES: c1ccc(N(c2ccc(-c3ccc(N(c4ccccc4)c4cccc5ccccc45)cc3)cc2)c2cccc3ccccc23)cc1] | U.S. Pat. No. 5,061,569 |
| | 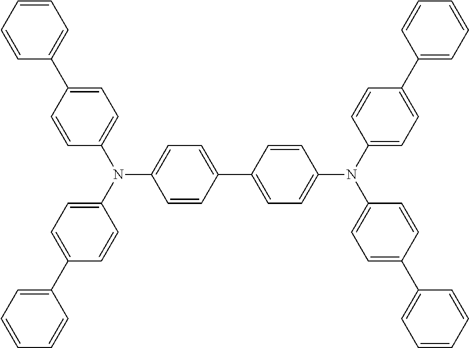 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1] | EP650955 |
| | 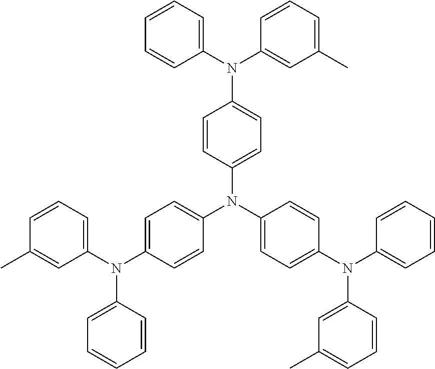 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1] | J. Mater. Chem. 3, 319 (1993) |
| | 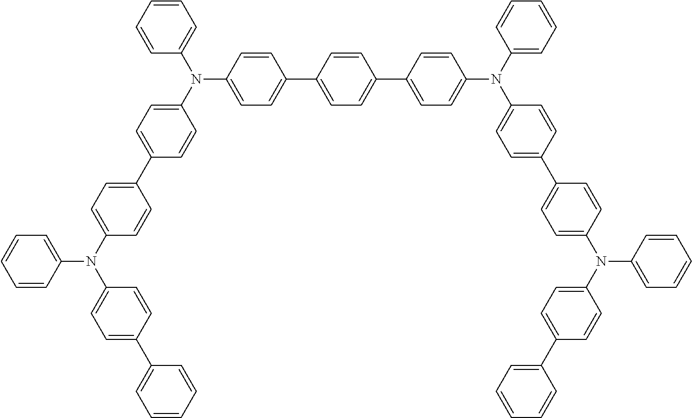 [SMILES: c1ccc(-c2ccc(N(c3ccccc3)c3ccc(-c4ccc(N(c5ccccc5)c5ccc(-c6ccc(-c7ccc(N(c8ccccc8)c8ccc(-c9ccc(N(c%10ccccc%10)c%10ccc(-c%11ccccc%11)cc%10)cc9)cc8)cc7)cc6)cc5)cc4)cc3)cc2)cc1] | Appl. Phys. Lett. 90, 183503 (2007) |
| | 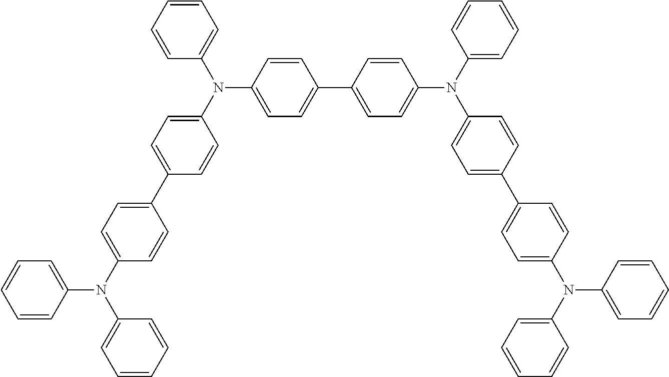 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1] | Appl. Phys. Lett. 90, 183503 (2007) |
| Triaylamine on spirofluorene core | 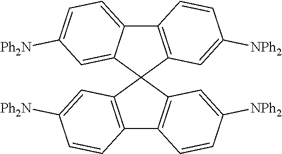 | Synth. Met. 91, 209 (1997) |
| Arylamine carbazole compounds | 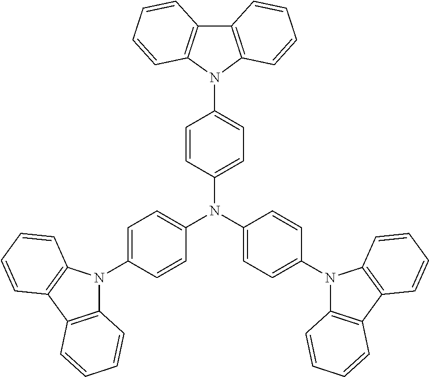 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1] | Adv. Mater. 6, 677 (1994), US20080124572 |
| Triarylamine with (di)benzothio- phene/ (di)benzofuran | 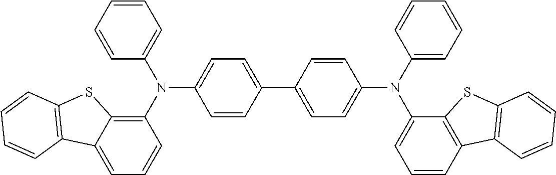 [SMILES: c1ccc(N(c2ccc(-c3ccc(N(c4ccccc4)c4cccc5c4sc4ccccc45)cc3)cc2)c2cccc3c2sc2ccccc23)cc1] | US20070278938, US20080106190 |
| Indolocarba- zoles | 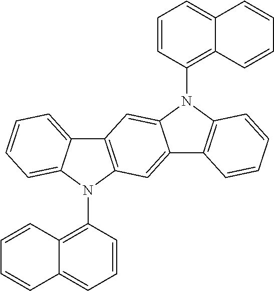 [SMILES: c1ccc2c(-n3c4ccccc4c4cc5c(cc43)c3ccccc3n5-c3cccc4ccccc34)cccc2c1] | Synth. Met. 111, 421 (2000) |
| Isoindole compounds | 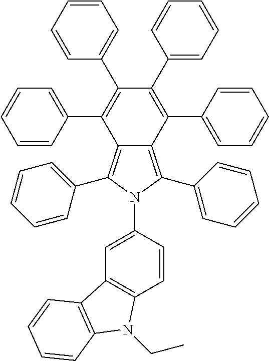 [SMILES: CCn1c2ccccc2c2cc(-n3c(-c4ccccc4)c4c(-c5ccccc5)c(-c5ccccc5)c(-c5ccccc5)c(-c5ccccc5)c4c3-c3ccccc3)ccc21] | Chem. Mater. 15, 3148 (2003) |
| Metal carbene complexes | 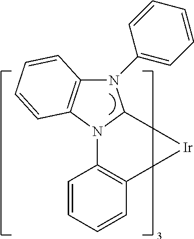 | US20080018221 |
| Phosphorescent | | |
| OLED host | | |
| materials | | |
| Red hosts | | |
| Arylcarbazoles | 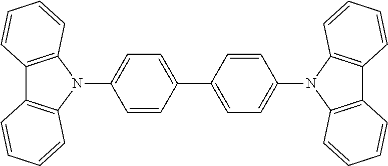 [SMILES: c1ccc2c(c1)c1ccccc1n2-c1ccc(-c2ccc(-n3c4ccccc4c4ccccc43)cc2)cc1] | Appl. Phys. Lett. 78, 1622 (2001) |
| Metal 8- hydroxy- quinolates (e.g., Alq₃, BAlq) | 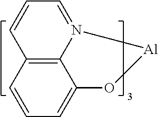 | Nature 395, 151 (1998) |
| | 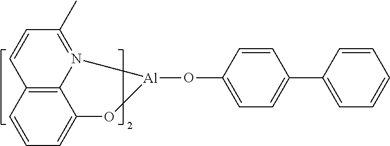 | US20060202194 |
| | 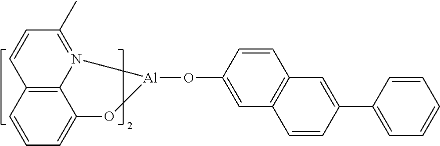 | WO2005014551 |
| | 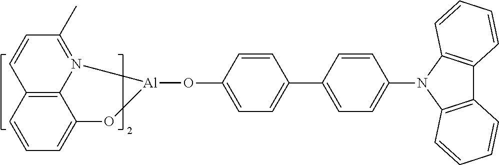 | WO2006072002 |
| Metal phenoxybenzo- thiazole compounds | 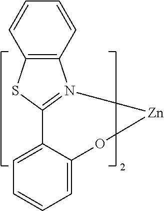 | Appl. Phys. Lett. 90, 123509 (2007) |
| Conjugated oligomers and polymers (e.g., polyfluorene) | 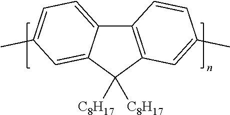 | Org. Electron. 1, 15 (2000) |
| Aromatic fused rings | 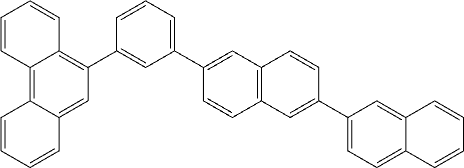 [SMILES: c1cc(-c2ccc3cc(-c4ccc5ccccc5c4)ccc3c2)cc(-c2cc3ccccc3c3ccccc23)c1] | WO2009066779, WO2009066778, WO2009063833, US20090045731, US20090045730, WO2009008311, US20090008605, US20090009065 |
| Zinc complexes | 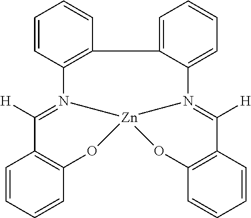 | WO2009062578 |
| Green hosts | | |
| Arylcarbazoles | 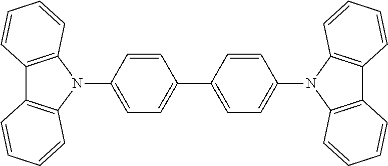 [SMILES: c1ccc2c(c1)c1ccccc1n2-c1ccc(-c2ccc(-n3c4ccccc4c4ccccc43)cc2)cc1] | Appl. Phys. Lett. 78, 1622 (2001) |
| | 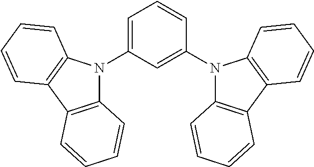 [SMILES: c1cc(-n2c3ccccc3c3ccccc32)cc(-n2c3ccccc3c3ccccc32)c1] | US20030175553 |
| | 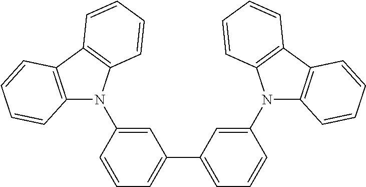 [SMILES: c1cc(-c2cccc(-n3c4ccccc4c4ccccc43)c2)cc(-n2c3ccccc3c3ccccc32)c1] | WO2001039234 |
| Aryltriphenyl- ene compounds | 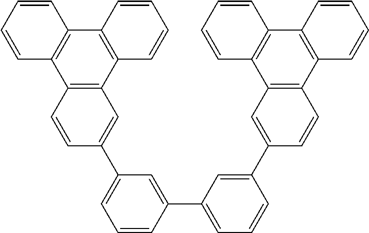 [SMILES: c1cc(-c2cccc(-c3ccc4c5ccccc5c5ccccc5c4c3)c2)cc(-c2ccc3c4ccccc4c4ccccc4c3c2)c1] | US20060280965 |
| | 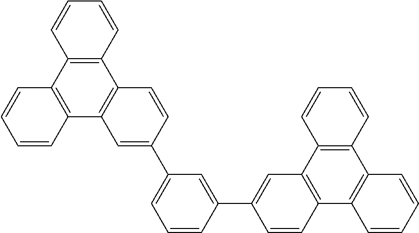 [SMILES: c1cc(-c2ccc3c4ccccc4c4ccccc4c3c2)cc(-c2ccc3c4ccccc4c4ccccc4c3c2)c1] | US20060280965 |
| | 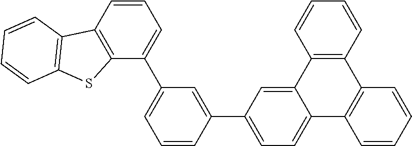 [SMILES: c1cc(-c2ccc3c4ccccc4c4ccccc4c3c2)cc(-c2cccc3c2sc2ccccc23)c1] | WO2009021126 |
| Donor acceptor type molecules | 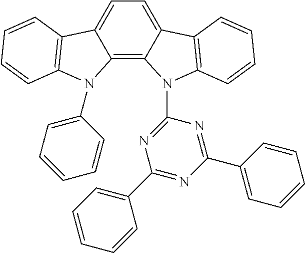 [SMILES: c1ccc(-c2nc(-c3ccccc3)nc(-n3c4ccccc4c4ccc5c6ccccc6n(-c6ccccc6)c5c43)n2)cc1] | WO2008056746 |
| Aza- carbazole/ DBT/DBF | 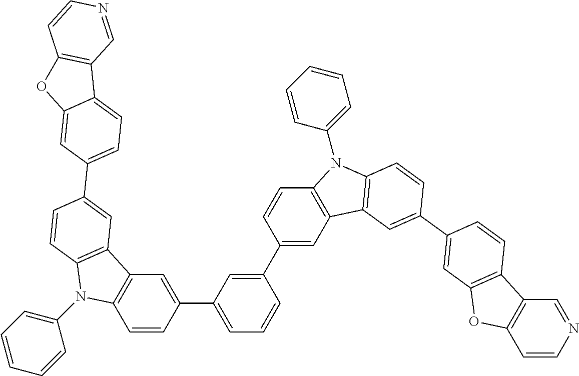 [SMILES: c1ccc(-n2c3ccc(-c4cccc(-c5ccc6c(c5)c5cc(-c7ccc8c(c7)oc7ccncc78)ccc5n6-c5ccccc5)c4)cc3c3cc(-c4ccc5c(c4)oc4ccncc45)ccc32)cc1] | JP2008074939 |
| Polymers (e.g., PVK) | 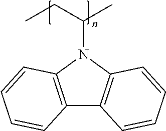 | Appl. Phys. Lett. 77, 2280 (2000) |
| Spirofluorene compounds | 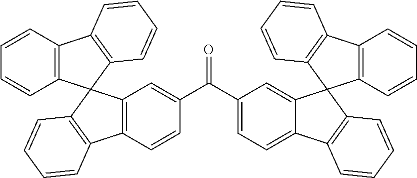 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2] | WO2004093207 |
| Metal phenoxybenzo- oxazole compounds | 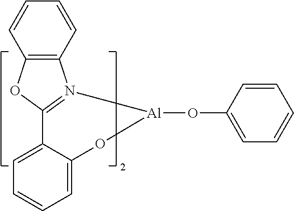 | WO2005089025 |
| | 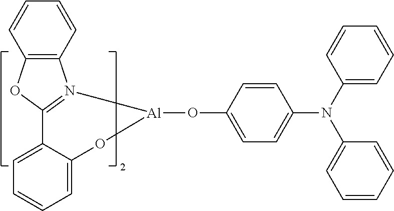 | WO2006132173 |
| | 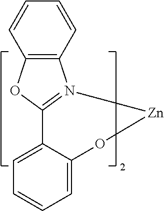 | JP200511610 |
| Spirofluorene- carbazole compounds | 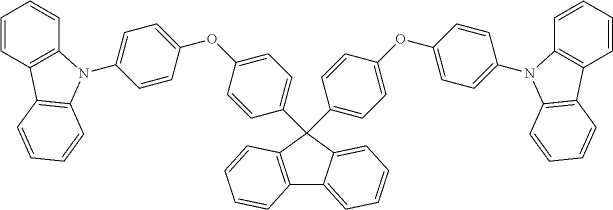 [SMILES: c1ccc2c(c1)-c1ccccc1C2(c1ccc(Oc2ccc(-n3c4ccccc4c4ccccc43)cc2)cc1)c1ccc(Oc2ccc(-n3c4ccccc4c4ccccc43)cc2)cc1] | JP2007254297 |
| | 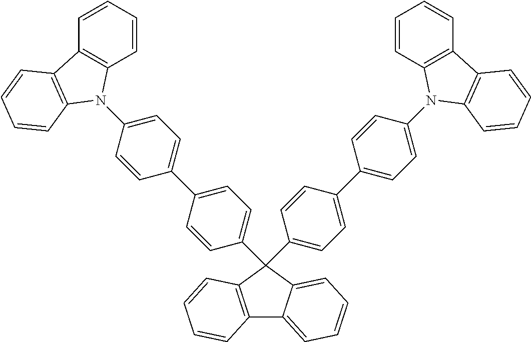 [SMILES: c1ccc2c(c1)-c1ccccc1C2(c1ccc(-c2ccc(-n3c4ccccc4c4ccccc43)cc2)cc1)c1ccc(-c2ccc(-n3c4ccccc4c4ccccc43)cc2)cc1] | JP2007254297 |
| Indolo- cabazoles | 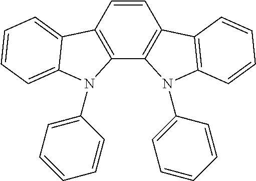 [SMILES: c1ccc(-n2c3ccccc3c3ccc4c5ccccc5n(-c5ccccc5)c4c32)cc1] | WO2007063796 |
| | 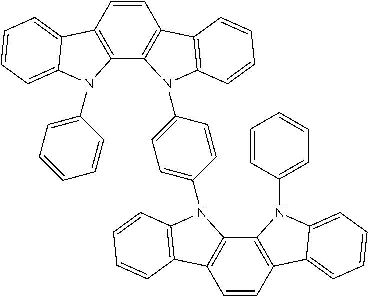 [SMILES: c1ccc(-n2c3ccccc3c3ccc4c5ccccc5n(-c5ccc(-n6c7ccccc7c7ccc8c9ccccc9n(-c9ccccc9)c8c76)cc5)c4c32)cc1] | WO2007063754 |
| 5-member ring electron deficient heterocycles (e.g., triazole, oxadiazole) | 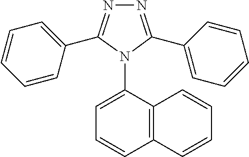 [SMILES: c1ccc(-c2nnc(-c3ccccc3)n2-c2cccc3ccccc23)cc1] | J. Appl. Phys. 90, 5048 (2001) |
| | 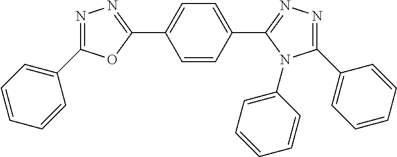 [SMILES: c1ccc(-c2nnc(-c3ccc(-c4nnc(-c5ccccc5)n4-c4ccccc4)cc3)o2)cc1] | WO2004107822 |
| Tetraphenylene complexes | 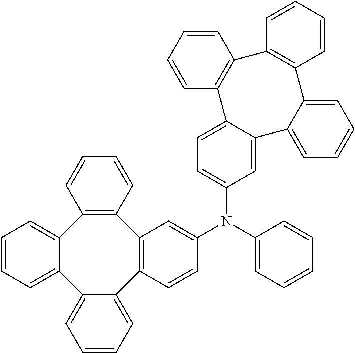 [SMILES: c1ccc(N(c2ccc3c(c2)-c2ccccc2-c2ccccc2-c2ccccc2-3)c2ccc3c(c2)-c2ccccc2-c2ccccc2-c2ccccc2-3)cc1] | US20050112407 |
| Metal phenoxy- pyridine compounds | 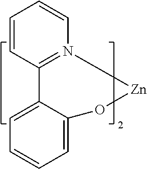 | WO2005030900 |
| Metal coordination complexes (e.g., Zn, Al with N\hat{ }N ligands) | 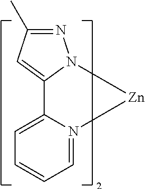 | US20040137268, US20040137267 |
| Blue hosts | | |
| Arylcarbazoles | 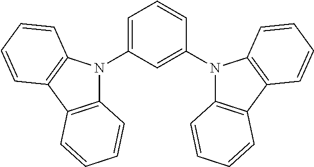 [SMILES: c1cc(-n2c3ccccc3c3ccccc32)cc(-n2c3ccccc3c3ccccc32)c1] | Appl. Phys. Lett. 82, 2422 (2003) |
| | 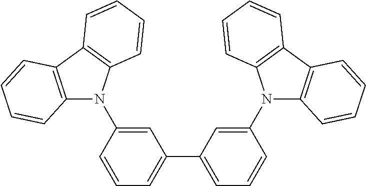 [SMILES: c1cc(-c2cccc(-n3c4ccccc4c4ccccc43)c2)cc(-n2c3ccccc3c3ccccc32)c1] | US20070190359 |
| Dibenzothio- phene/Dibenzo- furan-carbazole compounds | 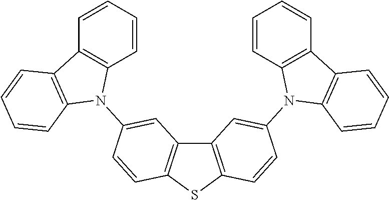 [SMILES: c1ccc2c(c1)c1ccccc1n2-c1ccc2sc3ccc(-n4c5ccccc5c5ccccc54)cc3c2c1] | WO2006114966, US20090167162 |
| | 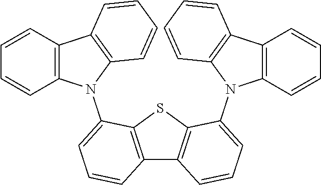 [SMILES: c1cc(-n2c3ccccc3c3ccccc32)c2sc3c(-n4c5ccccc5c5ccccc54)cccc3c2c1] | US20090167162 |
| | 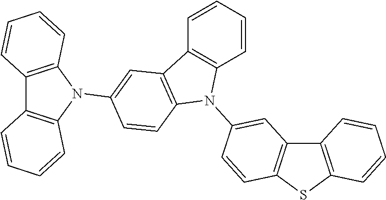 [SMILES: c1ccc2c(c1)sc1ccc(-n3c4ccccc4c4cc(-n5c6ccccc6c6ccccc65)ccc43)cc12] | WO2009086028 |
| | 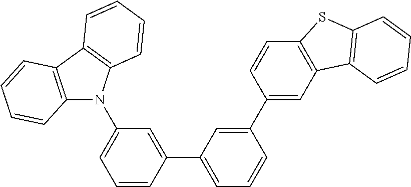 [SMILES: c1cc(-c2cccc(-n3c4ccccc4c4ccccc43)c2)cc(-c2ccc3sc4ccccc4c3c2)c1] | US20090030202, US20090017330 |
| Silicon aryl compounds | 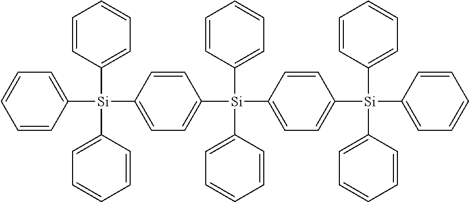 [SMILES: c1ccc([Si](c2ccccc2)(c2ccccc2)c2ccc([Si](c3ccccc3)(c3ccccc3)c3ccc([Si](c4ccccc4)(c4ccccc4)c4ccccc4)cc3)cc2)cc1] | US20050238919 |
| | 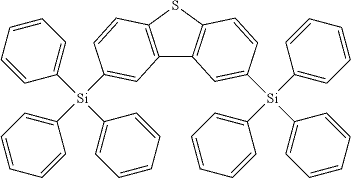 [SMILES: c1ccc([Si](c2ccccc2)(c2ccccc2)c2ccc3sc4ccc([Si](c5ccccc5)(c5ccccc5)c5ccccc5)cc4c3c2)cc1] | WO2009003898 |
| Silicon/ Germanium aryl compounds | 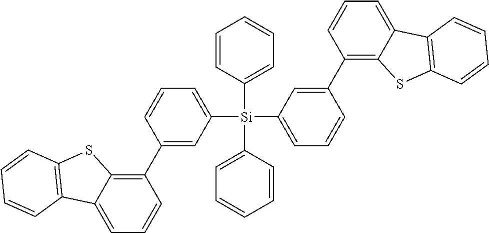 [SMILES: c1ccc([Si](c2ccccc2)(c2cccc(-c3cccc4c3sc3ccccc34)c2)c2cccc(-c3cccc4c3sc3ccccc34)c2)cc1] | EP2034538A |
| Aryl benzoyl ester | 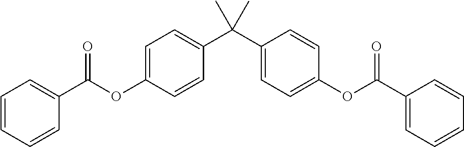 [SMILES: CC(C)(c1ccc(OC(=O)c2ccccc2)cc1)c1ccc(OC(=O)c2ccccc2)cc1] | WO2006100298 |
| High triplet metal organometallic complex | 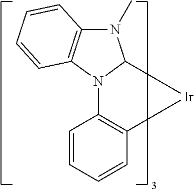 | U.S. Pat. No. 7,154,114 |
| Phosphorescent | | |
| dopants | | |
| Red dopants | | |
| Heavy metal porphyrins (e.g., PtOEP) | 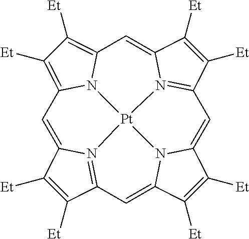 | Nature 395, 151 (1998) |
| Iridium(III) organometallic complexes | 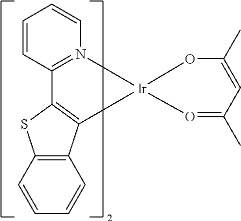 | Appl. Phys. Lett. 78, 1622 (2001) |
| | 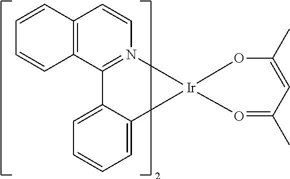 | US2006835469 |
| | 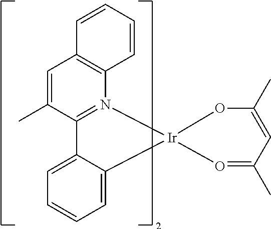 | US2006835469 |
| | 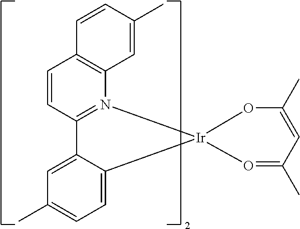 | US20060202194 |
| | 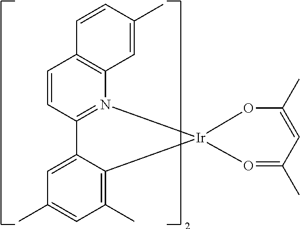 | US20060202194 |
| | 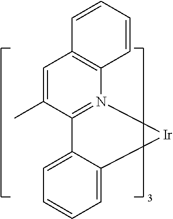 | US20070087321 |
| | 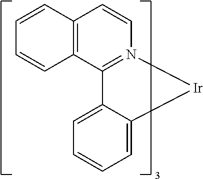 | US20070087321 |
| | 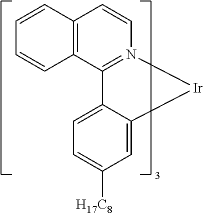 | Adv. Mater. 19, 739 (2007) |
| | 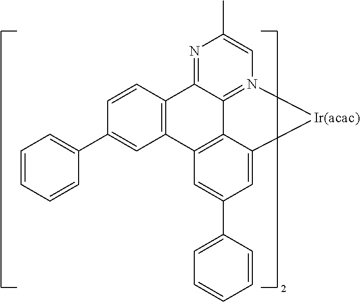 | WO2009100991 |
| | 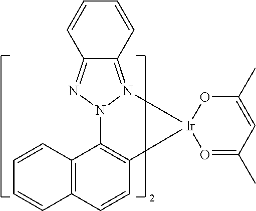 | WO2008101842 |
| Platinum(II) organometallic complexes | 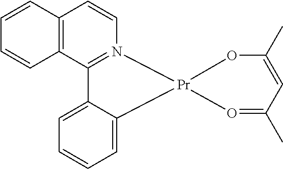 | WO2003040257 |
| Osminum(III) complexes | 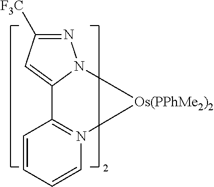 | Chem. Mater. 17, 3532 (2005) |
| Ruthenium(III) complexes | 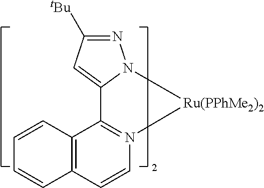 | Adv. Mater. 17, 1059 (2005) |
| Rhenium (I), (II), and (III) complexes | 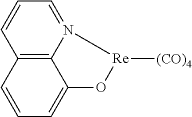 | US20050244673 |
| Green dopants | | |
| Iridium(III) organometallic complexes | 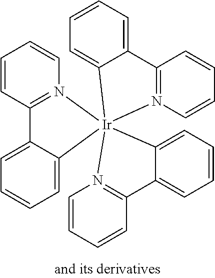 | Inorg. Chem. 40, 1704 (2001) |
| | 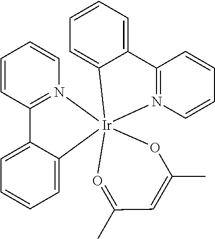 | US20020034656 |
| | 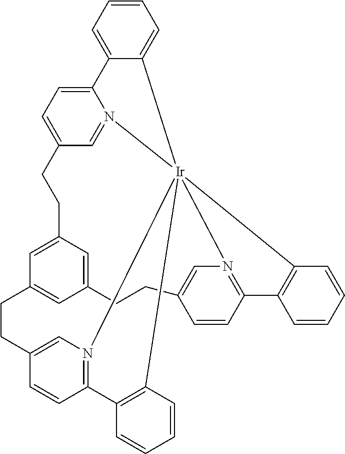 | U.S. Pat. No. 7,332,232 |
| | 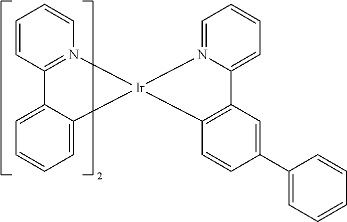 | US20090108737 |
| | 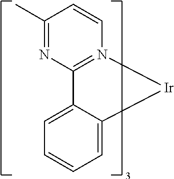 | US20090039776 |
| | 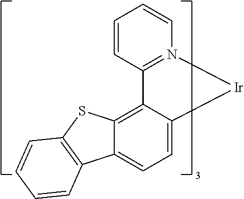 | U.S. Pat. No. 6,921,915 |
| | 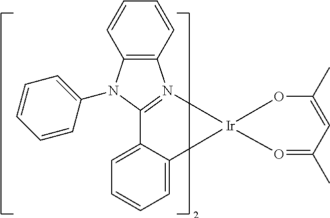 | U.S. Pat. No. 6,687,266 |
| | 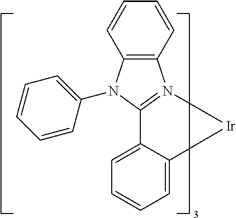 | Chem. Mater. 16, 2480 (2004) |
| | 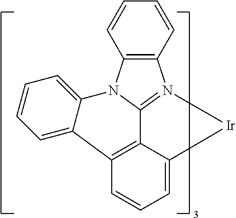 | US20070190359 |
| | 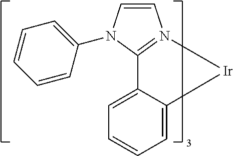 | US 20060008670 JP2007123392 |
| | 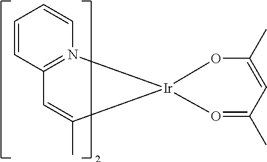 | Adv. Mater. 16, 2003 (2004) |
| | 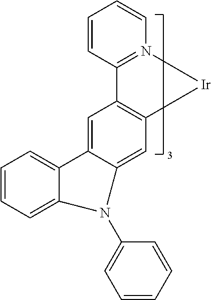 | Angew. Chem. Int. Ed. 2006, 45, 7800 |
| | 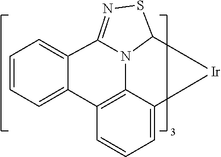 | WO2009050290 |
| | 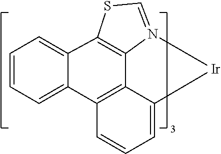 | US20090165846 |
| | 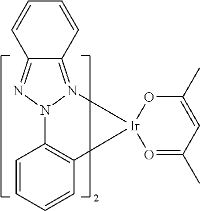 | US20080015355 |
| Monomer for polymeric metal organometallic compounds | 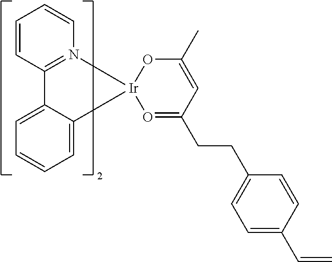 | U.S. Pat. No. 7,250,226, U.S. Pat. No. 7,396,598 |
| Pt(II) organometallic complexes, including polydentated ligands | 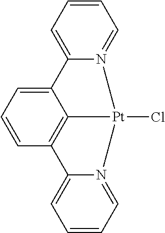 | Appl. Phys. Lett. 86, 153505 (2005) |
| | 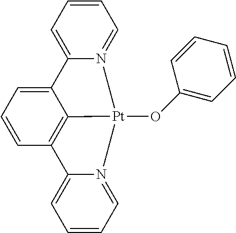 | Appl. Phys. Lett. 86, 153505 (2005) |
| | 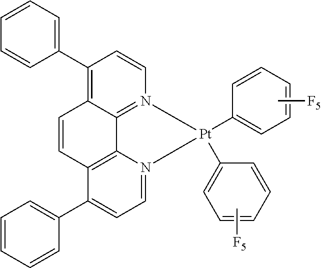 | Chem. Lett. 34, 592 (2005) |
| | 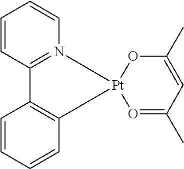 | WO2002015645 |
| | 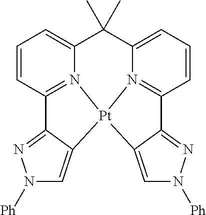 | US20060263635 |
| Cu complexes | 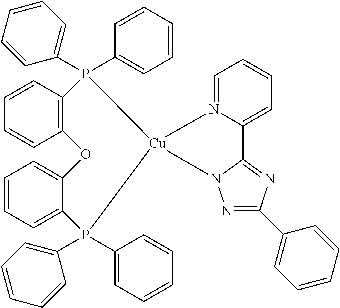 | WO2009000673 |
| Gold complexes | 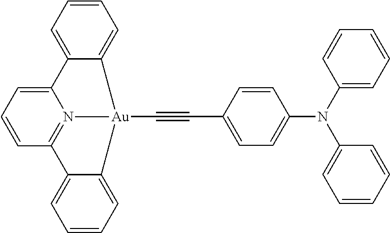 | Chem. Commun. 2906 (2005) |
| Rhenium(III) complexes | 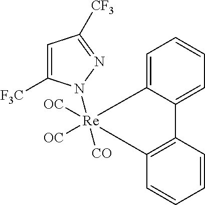 | Inorg. Chem. 42, 1248 (2003) |
| Deuterated organometallic complexes | 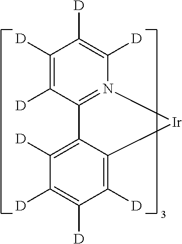 | US20030138657 |
| Organometallic complexes with two or more metal centers | 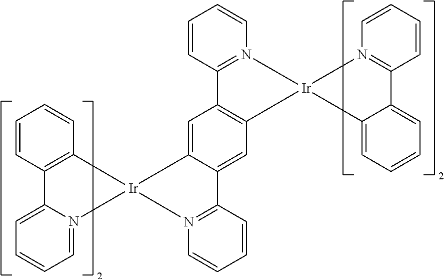 | US20030152802 |
| | 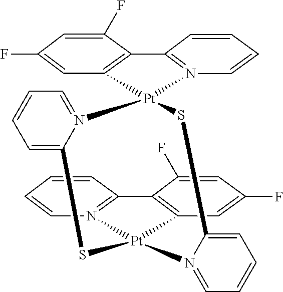 | U.S. Pat. No. 7,090,928 |
| Blue dopants | | |
| Iridium(III) organometallic complexes | 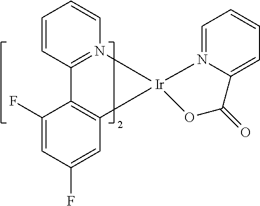 | WO2002002714 |
| | 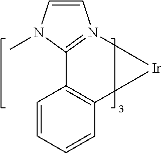 | WO2006009024 |
| | 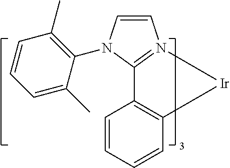 | US20060251923 |
| | 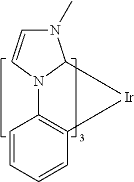 | U.S. Pat. No. 7,393,599, WO2006056418, US20050260441, WO2005019373 |
| | 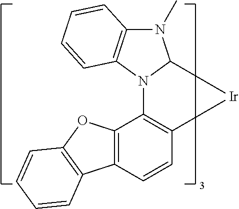 | U.S. Pat. No. 7,534,505 |
| | 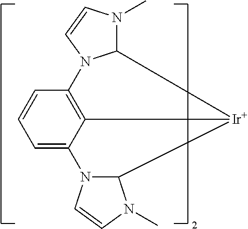 | U.S. Pat. No. 7,445,855 |
| | 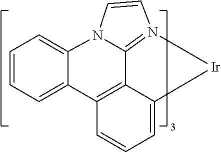 | US20070190359, US20080297033 |
| | 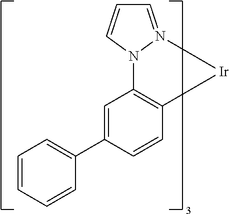 | U.S. Pat. No. 7,338.722 |
| | 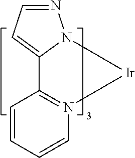 | US20020134984 |
| | 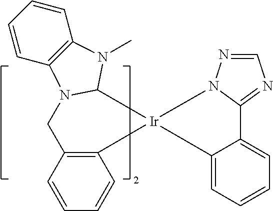 | Angew. Chem. Int. Ed. 47, 1 (2008) |
| | 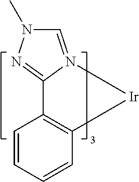 | Chem. Mater. 18, 5119 (2006) |
| | 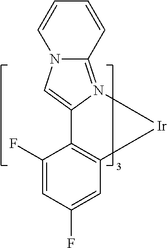 | Inorg. Chem. 46, 4308 (2007) |
| | 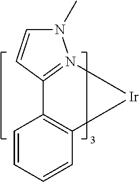 | WO2005123873 |
| | 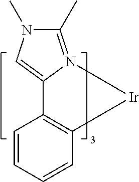 | WO2005123873 |
| | 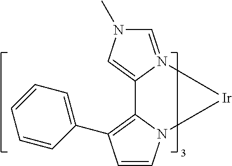 | WO2007004380 |
| | 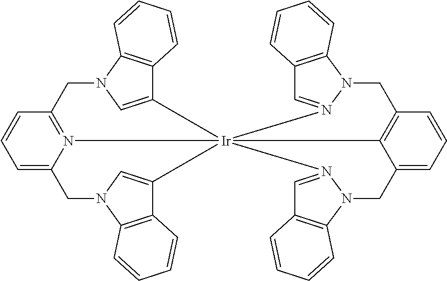 | OW2006082742 |
| Osmium(II) complexes | 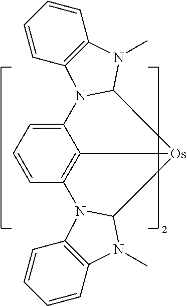 | U.S. Pat. No. 7,279,704 |
| | 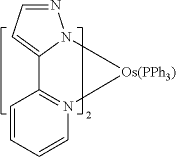 | Organometallics 23, 3745 (2004) |
| Gold complexes | 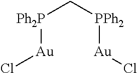 | Appl. Phys. Lett. 74, 1361 (1999) |
| Platinum(II) complexes | 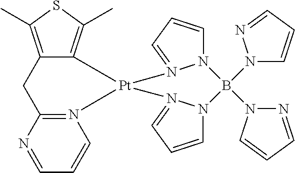 | WO2006098120, WO2006103874 |
| Exciton/hole | | |
| blocking layer | | |
| materials | | |
| Bathocuprine compounds (e.g., BCP, BPhen) | 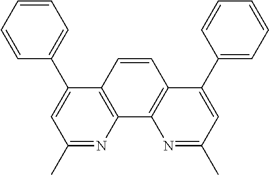 [SMILES: Cc1cc(-c2ccccc2)c2ccc3c(-c4ccccc4)cc(C)nc3c2n1] | Appl. Phys. Lett. 75, 4 (1999) |
| | 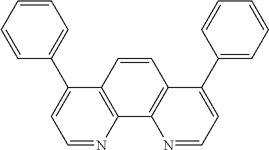 [SMILES: c1ccc(-c2ccnc3c2ccc2c(-c4ccccc4)ccnc23)cc1] | Appl. Phys. Lett. 79, 449 (2001) |
| Metal 8- hydroxy- quinolates (e.g., BAlq) | 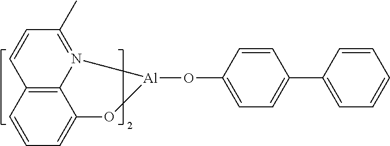 | Appl. Phys. Lett. 81, 162 (2002) |
| 5-member ring electron deficient heterocycles such as triazole, oxadiazole, imidazole, benzoimidazole | 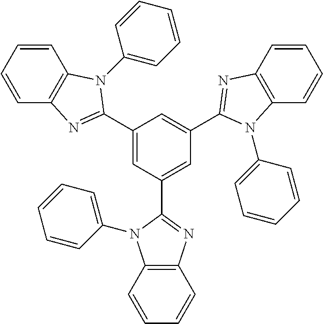 [SMILES: c1ccc(-n2c(-c3cc(-c4nc5ccccc5n4-c4ccccc4)cc(-c4nc5ccccc5n4-c4ccccc4)c3)nc3ccccc32)cc1] | Appl. Phys. Lett. 81, 162 (2002) |
| Triphenylene compounds | 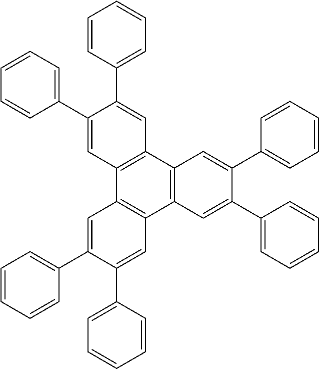 [SMILES: c1ccc(-c2cc3c4cc(-c5ccccc5)c(-c5ccccc5)cc4c4cc(-c5ccccc5)c(-c5ccccc5)cc4c3cc2-c2ccccc2)cc1] | US20050025993 |
| Fluorinated aromatic compounds | 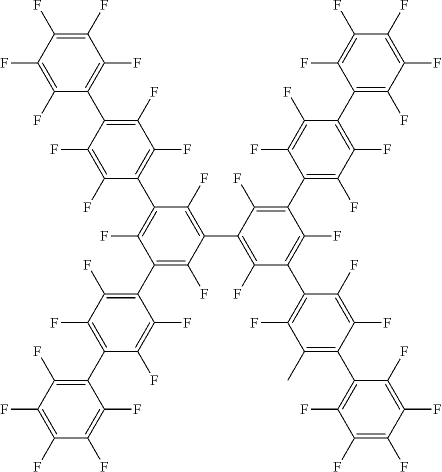 [SMILES: Cc1c(F)c(-c2c(F)c(-c3c(F)c(F)c(-c4c(F)c(F)c(F)c(F)c4F)c(F)c3F)c(F)c(-c3c(F)c(-c4c(F)c(F)c(-c5c(F)c(F)c(F)c(F)c5F)c(F)c4F)c(F)c(-c4c(F)c(F)c(-c5c(F)c(F)c(F)c(F)c5F)c(F)c4F)c3F)c2F)c(F)c(F)c1-c1c(F)c(F)c(F)c(F)c1F] | Appl. Phys. Lett. 79, 156 (2001) |
| Phenothiazine- S-oxide | 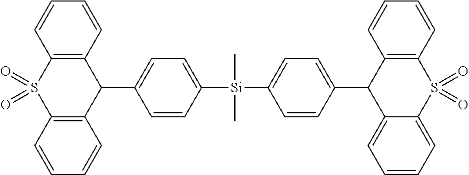 [SMILES: C[Si](C)(c1ccc(C2c3ccccc3S(=O)(=O)c3ccccc32)cc1)c1ccc(C2c3ccccc3S(=O)(=O)c3ccccc32)cc1] | WO2008132085 |
| Electron | | |
| trnasporting | | |
| materials | | |
| Anthracene- benzoimidazole compounds | 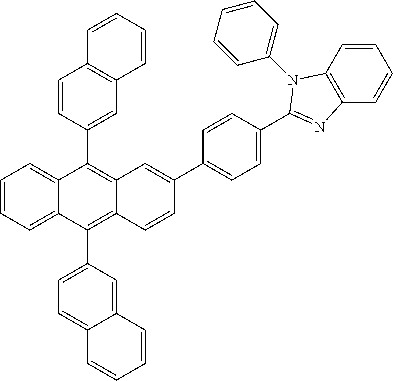 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1] | WO2003060956 |
| | 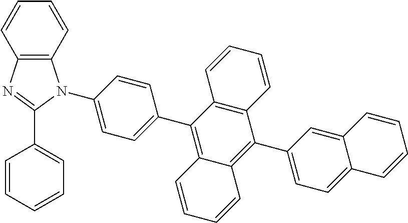 [SMILES: c1ccc(-c2nc3ccccc3n2-c2ccc(-c3c4ccccc4c(-c4ccc5ccccc5c4)c4ccccc34)cc2)cc1] | US20090179554 |
| Aza triphenylene derivatives | 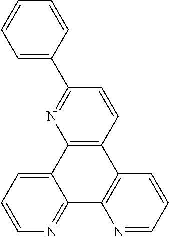 [SMILES: c1ccc(-c2ccc3c4cccnc4c4ncccc4c3n2)cc1] | US2009115316 |
| Anthracene- benzothiazole compounds | 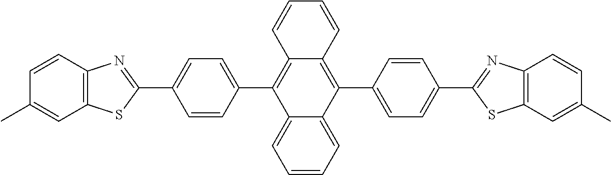 | Appl. Phys. Lett. 89, 063504 (2006) |
| Metal 8- hydroxy- quinolates (e.g., Alq₃, Zrq₄) | 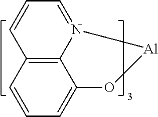 | Appl. Phys. Lett. 51, 913 (1987) U.S. Pat. No. 7,230,107 |
| Metal hydroxy- benoquinolates | 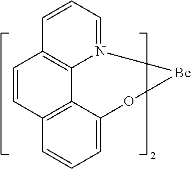 | Chem. Lett. 5, 905 (1993) |
| Bathocuprine compounds such as BCP, BPhen, etc | 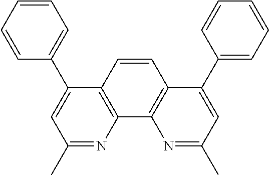 [SMILES: Cc1cc(-c2ccccc2)c2ccc3c(-c4ccccc4)cc(C)nc3c2n1] | Appl. Phys. Lett. 91, 263503 (2007) |
| | 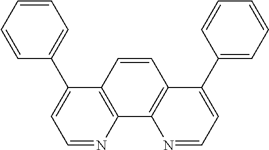 [SMILES: c1ccc(-c2ccnc3c2ccc2c(-c4ccccc4)ccnc23)cc1] | Appl. Phys. Lett. 79, 449 (2001) |
| 5-member ring electron deficient heterocycles (e.g., triazole, oxadiazole, imidazole, benzoimida- zole) | 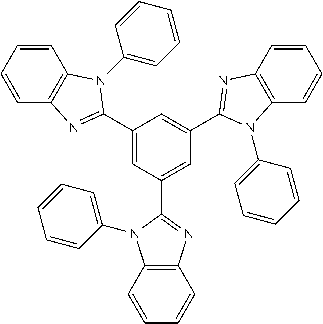 [SMILES: c1ccc(-n2c(-c3cc(-c4nc5ccccc5n4-c4ccccc4)cc(-c4nc5ccccc5n4-c4ccccc4)c3)nc3ccccc32)cc1] | Appl. Phys. Lett. 74, 865 (1999) |
| | 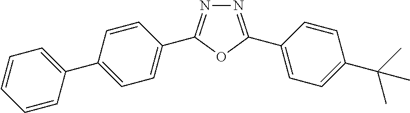 [SMILES: CC(C)(C)c1ccc(-c2nnc(-c3ccc(-c4ccccc4)cc3)o2)cc1] | Appl. Phys. Lett. 55, 1489 (1989) |
| | 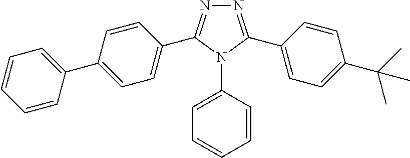 [SMILES: CC(C)(C)c1ccc(-c2nnc(-c3ccc(-c4ccccc4)cc3)n2-c2ccccc2)cc1] | Jpn. J. Apply. Phys. 32, L917 (1993) |
| Silole compounds | 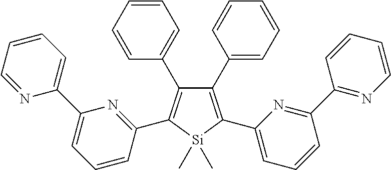 [SMILES: C[Si]1(C)C(c2cccc(-c3ccccn3)n2)=C(c2ccccc2)C(c2ccccc2)=C1c1cccc(-c2ccccn2)n1] | Org. Electron. 4, 113 (2003) |
| Arylborane compounds | 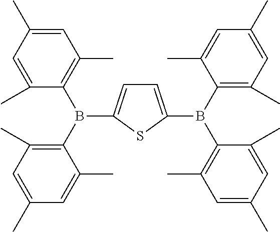 | J. Am. Chem. Soc. 120, 9714 (1998) |
| Fluorinated aromatic compounds | 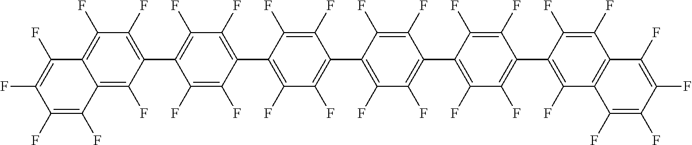 [SMILES: Fc1c(F)c(-c2c(F)c(F)c(-c3c(F)c(F)c4c(F)c(F)c(F)c(F)c4c3F)c(F)c2F)c(F)c(F)c1-c1c(F)c(F)c(-c2c(F)c(F)c(-c3c(F)c(F)c4c(F)c(F)c(F)c(F)c4c3F)c(F)c2F)c(F)c1F] | J. Am. Chem. Soc. 122, 1832 (2000) |
| Fullerene (e.g., C60) | 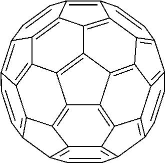 [SMILES: c1cc2c3ccc4c5ccc6c7ccc8c9ccc%10c1c1c2c2c3c4c3c5c6c4c7c8c5c9c%10c1c1c2c3c4c51] | US20090101870 |
| Triazine complexes | 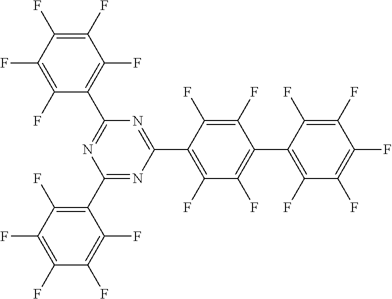 [SMILES: Fc1c(F)c(F)c(-c2nc(-c3c(F)c(F)c(F)c(F)c3F)nc(-c3c(F)c(F)c(-c4c(F)c(F)c(F)c(F)c4F)c(F)c3F)n2)c(F)c1F] | US20040036077 |
| Zn (N\hat{ }N) complexes | 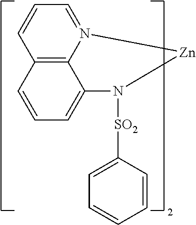 | U.S. Pat. No. 6,528,187 |

### Experimental

Chemical abbreviations used throughout this document are as follows: Cy is cyclohexyl, dba is dibenzylideneacetone, EtOAc is ethyl acetate, DME is dimethoxyethane, dppe is 1,2-bis(diphenylphosphino)ethane, THF is tetrahydrofuran, DCM is dichloromethane, S-Phos is dicyclohexyl(2′,6′-dimethoxy-[1,1′-biphenyl]-2-yl)phosphine.

Exemplary Dehalogenation Reaction 1

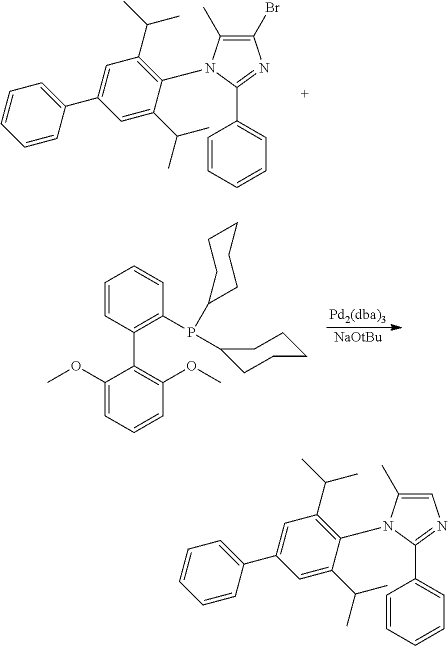

4-Bromo-1-(3,5-diisopropyl-[1,1′-biphenyl]-4-yl)-5-methyl-2-phenyl-1H-imidazole (6.9 g, 14.6 mmol), Pd₂(dba)₃ (0.13 g, 0.15 mmol), dicyclohexyl(2′,6′-dimethoxy-[1,1′-biphenyl]-2-yl)phosphine (0.24 g, 0.58 mmol) and sodium tert-butoxide (2.8 g, 29.1 mmol) were charged into a 500 mL 2-neck flask with 2-propanol (250 mL) and the mixture was degassed and back-filled with nitrogen before being heated to reflux overnight. After removal of the solvent under reduced pressure, 250 mL of water was added and the resultant mixture extracted twice with 300 mL of ethyl acetate. The combined organic layers were washed twice with 100 mL of aqueous 10% LiCl. The extracts were dried over magnesium sulfate, filtered, and the solvent was removed under vacuum. The crude product was purified first on silica gel with 20/10/70 (v/v) THF/DCM/hexanes followed by chromatography in neutral alumina with 20-30% ethyl acetate in hexanes to yield 5.4 g (80%) of a white solid, which was then recrystallized from 75 mL of hexanes to yield 4.6 g of debrominated product as a white solid (HPLC purity: 99.9%). Absence of bromine was confirmed by GC/MS and NMR.

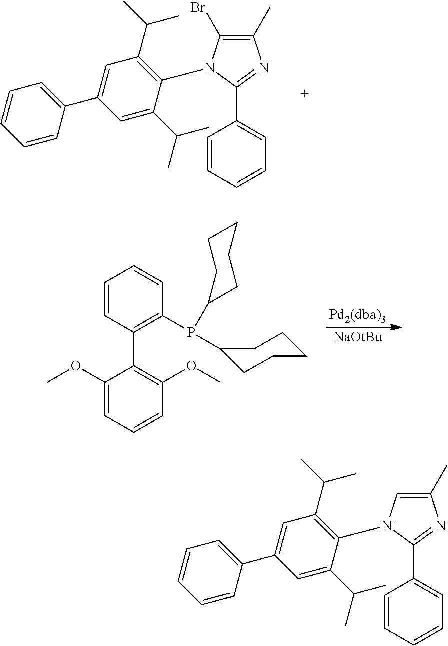

5-Bromo-1-(3,5-diisopropyl-[1,1′-biphenyl]-4-yl)-4-methyl-2-phenyl-1H-imidazole (3.0 g, 6.3 mmol), Pd₂(dba)₃ (0.12 g, 0.13 mmol), dicyclohexyl(2′,6′-dimethoxy-[1,1′-biphenyl]-2-yl)phosphine (0.21 g, 0.51 mmol), and sodium tert-butoxide (1.2 g, 12.7 mmol) were mixed in 50 mL of isopropanol. The mixture was degassed with nitrogen for 20 min. before being heated to reflux overnight. After removal of the solvent under reduced pressure, dichloromethane (100 mL) was added. The dichloromethane solution was washed with water (2×100 mL) and dried over MgSO₄. After filtration and removal of the solvent, the crude material was chromatographed on silica with 10/90 (v/v) ethyl acetate/hexanes and recrystallized twice from hexanes to give 2.3 g of the debrominated product as a white solid. Absence of bromine was confirmed by GC-MS and NMR.

### Exemplary Dehalogenation Reaction 2

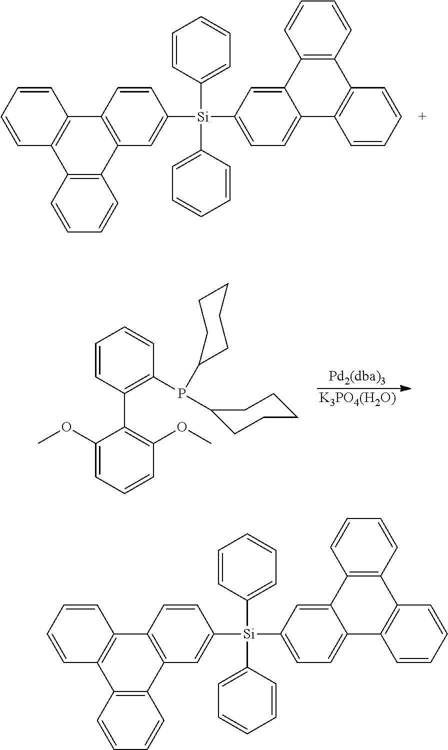

Diphenyldi(triphenylen-2-yl)silane (9.6 g, 15.07 mmol) (98.9% containing 1.1% of a brominated impurity), dicyclohexyl(2′,6′-dimethoxy-[1,1′-biphenyl]-2-yl)phosphine (0.495 g, 1.206 mmol) and potassium phosphate tribasic monohydrate (6.94 g, 30.1 mmol) were suspended in m-xylene (500 mL) and water (50 mL) and degassed by bubbling nitrogen through the mixture. Pd₂(dba)₃ (0.276 g, 0.301 mmol) was then added and the mixture heated to reflux. Aliquots of the reaction mixture were taken at regular intervals and checked by HPLC (Table 1).

TABLE 1
Percentage halogenated impurity as a function of time.

| | Percent brominated |
|---|---|
| Time (hours) | impurity (%) |
| 24 | 0.073 |
| 36 | 0.056 |
| 72 | 0.048 |
| 96 | 0.029 |

After cooling to room temperature, the crude reaction mixture was poured through a plug of Celite® on a glass frit, and washed with DCM. Removal of the solvent gave 11.3 g of a dark red solid, which was then filtered through a plug of silica on a frit. Removal of the solvent gave 9.7 g of a tan solid. The crude material was chromatographed on silica with 7-6:3-4 hexane/DCM (v/v) and recrystallized by slow evaporation of DCM from a ˜3:1 hexanes:DCM (v/v) solution to give 8.3 g of product with 99.91% purity by HPLC (0.029% brominated impurity).

### COMPARATIVE EXAMPLES

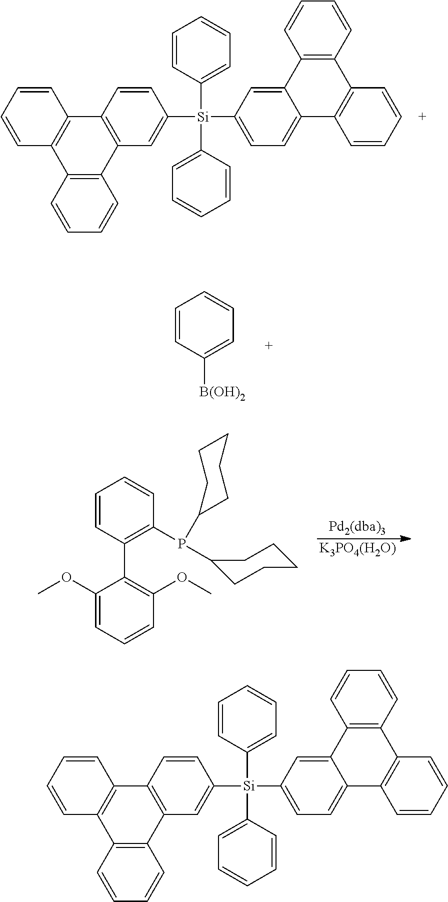

### Comparative Example 1

Diphenyldi(triphenylen-2-yl)silane (1.0 g, 1.6 mmol) (98.9% containing 1.1% of a brominated impurity) was suspended in toluene (50 mL) and water (5 mL). Phenylboronic acid (0.19 g, 1.57 mmol), dicyclohexyl(2′,6′-dimethoxy-[1,1′-biphenyl]-2-yl)phosphine (0.052 g, 0.126 mmol) and potassium phosphate tribasic monohydrate (0.72 g, 3.1 mmol) were added and the reaction mixture degassed by bubbling nitrogen. Pd₂(dba)₃ (0.029 g, 0.031 mmol) was then added and the mixture heated to reflux for 20 h. After cooling to room temperature, the solvent was removed under reduced pressure and the resulting residue was dissolved in DCM and extracted twice with water. The organic layer was dried over sodium sulfate, filtered, and the solvent removed under reduced pressure. Thin layer chromatography (TLC) in 7/3 (v/v) hexane/DCM showed only one spot. The crude material was chromatographed on silica gel with 7/3 (v/v/) hexane/DCM and recrystallized by slow evaporation of DCM from a ˜3:1 hexanes:DCM solution to give 0.47 g of a white solid. HPLC analysis of the white solid indicated that the brominated impurity was absent and two new peaks were observed. The overall purity of the product was found to be 99.2%, but the impurities were inseparable on silica gel.

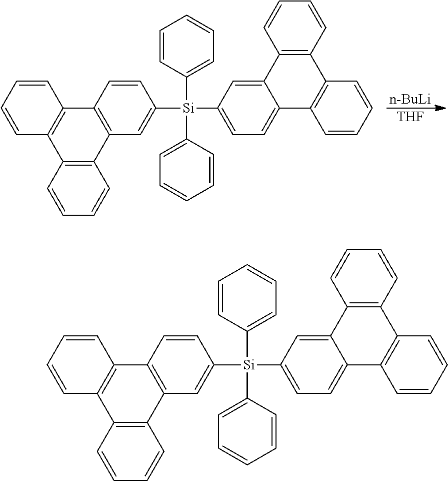

### Comparative Example 2

Diphenyldi(triphenylen-2-yl)silane (3.0 g, 4.7 mmol) (98.6% containing 1.4% of a brominated impurity) was suspended in THF (200 mL) and the solution cooled to −70° C. n-BuLi (2.5 Min hexane, 1.8 mL, 4.7 mmol) was then added dropwise and the reaction mixture was stirred for 3 h at −78° C. The reaction mixture was then allowed to warm to −30° C. and quenched with MeOH and water. After warming to room temperature, DCM was added and the mixture extracted twice with water (200 mL). The organic layer was dried over sodium sulfate, filtered, and the solvent removed under reduced pressure to yield 2.6 g of a white solid. The HPLC indicated that the brominated impurity was still present. The overall purity of the product was found to be 98.7%, with about 1.3% of brominated impurity.

### Comparative Example 3

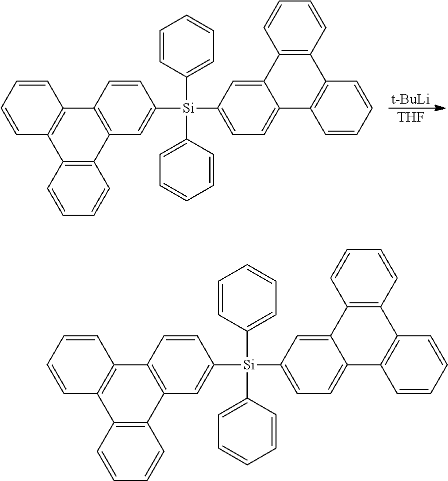 [SMILES: "t-BuLi" ; c1ccc([Si](c2ccccc2)(c2ccc3c4ccccc4c4ccccc4c3c2)c2ccc3c4ccccc4c4ccccc4c3c2)cc1 ; c1ccc([Si](c2ccccc2)(c2ccc3c4ccccc4c4ccccc4c3c2)c2ccc3c4ccccc4c4ccccc4c3c2)cc1 ; "THF"]

Diphenyldi(triphenylen-2-yl)silane (3.1 g, 3.9 mmol) (98.6% containing 1.4% of a brominated impurity) was suspended in THF (200 mL) and the solution cooled to −70° C. t-BuLi (1.7 Min pentane, 2.3 mL, 3.9 mmol) was then added dropwise and the reaction mixture was stirred for 4 h at −70° C. The reaction mixture was then quenched with MeOH and water and allowed to warm to room temperature. The solvents were removed under reduced pressure and water and ether were added. The resulting white solid was was filtered and washed with water, then ether to yield 3.1 g of a white solid. The HPLC indicated that the brominated impurity was still present. The overall purity of the product was found to be 99.3%, with about 0.7% of brominated impurity.

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

1. A method of reducing halogen content comprising:
A) reacting at least one aromatic halide with a transition metal complex by
1) oxidatively inserting the transition metal complex into a carbon-halogen bond in said aromatic halide to form an metal-carbon bonded species; and
2) converting the metal-carbon bonded species into a reaction product, wherein the reaction product has the halogen in the aromatic halide replaced by hydrogen;
B) purifying the reaction product to give a purified product;
wherein the oxidative insertion takes place in the presence of a base.

2. The method of claim 1, further comprising using the purified product in an organic electronic device.

3. The method of claim 1, further comprising:
depositing a first electrode;
depositing an organic layer comprising the purified product on the first electrode; and
depositing a second electrode on the organic layer.

4. The method of claim 1, wherein the purified product contains less than 300 ppm of halogen.

5. The method of claim 1, wherein the purified product contains less than 50 ppm of halogen.

6. The method of claim 1, wherein the purified product contains less than 10 ppm of halogen.

7. The method of claim 1, wherein the transition metal complex comprises a metal complex containing palladium, platinum, copper, or nickel.

8. The method of claim 1, wherein the aromatic halide comprises an imidazole, a pyridine, a quinoline, an isoquinoline, an aldehyde, a ketone, an alcohol, or combinations thereof.

9. The method of claim 1, wherein the aromatic halide comprises a triphenylene, an aza-triphenylene, a carbazole, an aza-carbazole, a dibenzothiphene, a dibenzofuran, a dibenzoselenophene, an aza-dibenzothiophene, an aza-dibenzofuran, an aza-dibenzoselenophene, or combinations thereof.

10. The method of claim 1, wherein less than 10 ppm of the reaction product contains a new carbon-carbon bond.

11. The method of claim 1, wherein less than 90% by weight of the reaction product contains a new carbon-carbon bond.

12. The method of claim 1, wherein the aromatic halide is used in an electroluminescent device.

13. The method of claim 7, wherein the transition metal complex is a palladium complex.

14. The method of claim 13, wherein the palladium complex is a palladium(II) complex.

15. The method of claim 14, wherein the palladium(II) complex is selected from the group consisting of PdCl₂, Pd(PPh₃)₂Cl₂, Pd(OAc)₂, [Pd(allyl)Cl]₂, Pd(benzonitrile)₂Cl₂, Pd(dppf)Cl₂, and combinations thereof.

16. The method of claim 13, wherein the palladium complex includes one or more ligands selected from the group consisting of dibenzylideneacetone, 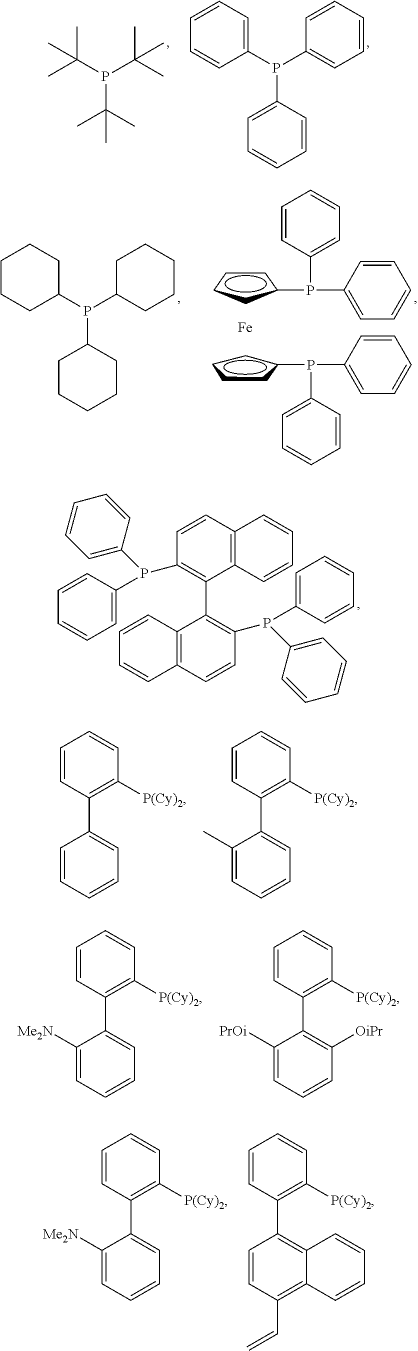 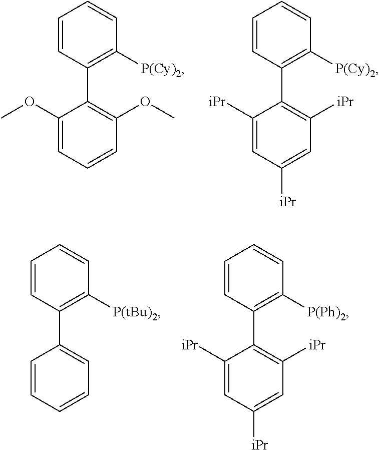
and combinations thereof.

17. The method of claim 1, wherein the base is an oxygen-containing base.

18. The method of claim 17, wherein the base is an alkali metal salt of a compound selected from the group consisting of an alkoxide, a phosphate, and a carbonate.

19. The method of claim 18, wherein the alkoxide is NaO^{t}Bu or KO^{t}Bu.

20. The method of claim 18, wherein the phosphate is K₃PO₄.

21. A first device comprising a first organic light emitting device, further comprising:
an anode;
a cathode; and
an organic layer, disposed between the anode and the cathode, wherein the organic layer is deposited from a source comprising a purified product formed by
A) reacting at least one aromatic halide with a transition metal complex by
1) oxidatively inserting the transition metal complex into a carbon-halogen bond in said aromatic halide to form an metal-carbon bonded species; and
2) converting the metal-carbon bonded species into a reaction product, wherein the reaction product has the halogen in the aromatic halide replaced by hydrogen;
B) purifying the reaction product to give the purified product;
wherein the oxidative insertion takes place in the presence of a base.

22. The first device of claim 21, wherein the organic layer has a halogen content of less than 300 ppm.
